(19) United States
(12) Patent Application Publication
(10) Pub. No.: US 2006/0286167 A1
(43) Pub. Date: Dec. 21, 2006

(54) Compositions and methods for the treatment of neurodegenerative diseases

(76) Inventors: Jane Staunton, Roslindale, MA (US); Xiaowei Jin, Cambridge, MA (US); Dina Solimini Rufo, South Weymouth, MA (US); Michael Monteiro, Malden, MA (US)

Correspondence Address: CLARK & ELBING LLP, 101 FEDERAL STREET, BOSTON, MA, 02110, US

(21) Appl. No.: 11/415,926

(22) Filed: May 2, 2006

Related U.S. Application Data
(60) Provisional application No. 60/677,022, filed on May 2, 2005.
(60) Provisional application No. 60/698,184, filed on Jul. 11, 2005.
(60) Provisional application No. 60/761,573, filed on Jan. 24, 2006.

(51) Int. Cl. A61K 31/551; A61K 31/375; A61K 9/20; A61K 31/195

(52) U.S. Cl. 424/464; 514/218; 514/561; 514/474

(57) ABSTRACT

The invention features compositions and methods for the treatment of neurodegenerative diseases.

### CROSS-REFERENCE TO RELATED APPLICATIONS

This application claims benefit from U.S. Provisional Application Nos. 60/677,022, filed May 2, 2005, 60/698,184, filed Jul. 11, 2005, and 60/761,573, filed Jan. 24, 2006, each of which is hereby incorporated by reference.

### BACKGROUND OF THE INVENTION

Proximal Spinal Muscular Atrophy (SMA), a common genetic cause of infant mortality, is an autosomal recessive disorder in which alpha motor neuron death in the spinal cord is observed. The primary genetic lesion that causes SMA is a deletion or mutation of the telomeric copy of the survival motor neuron gene (SMN1). The centromeric survival motor-neuron gene (SMN2), a hypofunctional allele of SMN1, is unaffected in the disease. This information has lead to the generation of a mouse model of SMA, in which the single mouse SMN gene is deleted and the resulting embryonic lethality is suppressed by introduction of the human SMN2 transgene. SMN is a 38 kDa protein ubiquitously expressed in both cytoplasm and nuclei. In the nucleus, SMN is found in gemini of coiled bodies (gems), named for their association with coiled bodies. SMN associates with itself and forms a complex with a series of proteins, including the Sm proteins, SIP-1 (gemin 2), gemin 3 and gemin 4 and possibly other proteins. This SMN-containing complex functions in snRNP biogenesis, participating in pre-mRNA splicing in the nucleus. A series of other proteins have been reported to interact with SMN, including profilins, E2 and FUSE, suggesting other possible roles for SMN. Despite the insights derived from identification of SMN as the principal genetic cause of SMA, the detailed molecular pathogenesis of the disease remains enigmatic. The basis for selective death of alpha motor neurons compared to other cell types in SMA patients and mice is not understood.

The primary molecular defect in most patients with SMA is decreased SMN protein levels. This deficiency results in the selective degeneration of lower motor neurons and the loss of motor function, and is frequently fatal. Small molecules that increase the amount of SMN protein in cells are much sought after for their potential therapeutic value to SMA patients. Previous screens and research efforts have been directed towards discovering small molecules that alter splicing of the SMN2 pre-mRNA, or of compounds that activate the SMN2 promoter. However, many of these compounds do not increase the amount of SMN protein in cells by a significant amount. In addition, most of the identified compounds show toxicities that limit their therapeutic suitability.

Thus there is a need for agents that may be used to treat SMA and other neurodegenerative diseases.

### SUMMARY OF THE INVENTION

We have identified compounds that increase SMN levels in SMA patient fibroblasts in vitro. These compounds may be used alone or in combination for the treatment of SMA or another neurodegenerative disease.

Accordingly, in a first aspect, the invention features a composition that includes: (a) a first agent selected from the agents of Table 1; and (b) a second agent useful for treating a neurodegenerative disease. Desirably, the first agent and the second agent are present in amounts that, when administered to a patient, are sufficient to treat a neurodegenerative disease or increase SMN protein levels (i.e., result in a statistically significant increase in SMN protein levels compared to a control). The composition optionally includes one or more additional agents selected from Table 1. The composition may be formulated for oral or systemic administration. In certain embodiments, the two agents are ascorbic acid and memantine; ascorbic acid and indoprofen; ascorbic acid and amantadine; ascorbic acid and guanfacine; ubenimex and amantadine; amrinone and memantine; amrinone and amantadine; amrinone and indoprofen; amrinone and guanfacine; guanfacine and memantine; gunafacine and amantadine; alosetron and memantine; alosetron and amantadine; or indoprofen and memantine.

The invention also features a method for treating a neurodegenerative disease or increasing SMN protein levels in a patient having SMA by administering to a patient in need thereof one, two, or more agents selected from the agents of Table 1 in an amount sufficient to treat the neurodegenerative disease or increase SMN protein levels. If two or more agents are administered, it is desirable that the agents be administered simultaneously or within 28 days, 14 days, 10, days, 7 days, or 24 hour of each other, or simultaneously, in amounts that together are sufficient to treat the neurodegenerative disease or increase SMN protein levels. In certain embodiments, the two agents are ascorbic acid and memantine; ascorbic acid and indoprofen; ascorbic acid and amantadine; ascorbic acid and guanfacine; ubenimex and amantadine; amrinone and memantine; amrinone and amantadine; anrinone and indoprofen; amrinone and guanfacine; guanfacine and memantine; gunafacine and amantadine; alosetron and memantine; alosetron and amantadine; or indoprofen and memantine.

The method may further include the step of administering to the patient one or more additional therapeutic agent useful for treating a neurodegenerative disease, such as those described herein. If the patient is administered more than one agent, the different agents may be admixed together in a single formulation. When administered in separate formulations, the agents may be administered simultaneously or within 14 days, 7 days, or 1 day of each other. These agents may or may not be administered by the same route of administration (e.g., oral, intravenous, intramuscular, ophthalmic, topical, dermal, subcutaneous, and rectal). If desired, an agent maybe administered at a high dosage, low dosage.

The invention also features kits for treating neurodegenerative diseases.

In one version, the kit includes (i) an agent selected from the agents of Table 1; and (ii) instructions for administering the agent to a patient having a neurodegenerative disease.

In another version, the kit includes (i) a composition containing two agents selected from the agents of Table 1; and (ii) instructions for administering the composition to a patient having a neurodegenerative disease.

Yet another kit includes (i) a first agent selected from the agents of Table 1; (ii) a second agent selected from the agents of Table 1; and (iii) instructions for administering the first and second agents to a patient having a neurodegenerative disease.

Still another kit includes (i) an agent selected from the agents of Table 1; and (ii) instructions for administering the agent with a second agent selected from the agents of Table 1 to a patient having a neurodegenerative disease.

Another kit of the invention includes (i) a composition containing (a) a first agent selected from the agents of Table 1; and (b) a second agent that is useful for treating a neurodegenerative disease; and (ii) instructions for administering the composition to a patient having a neurodegenerative disease.

Yet another kit of the invention includes (i) a first agent selected from the agents of Table 1; (ii) a second agent that is useful for treating a neurodegenerative disease; and (iii) instructions for administering the first and second agents to a patient having a neurodegenerative disease.

Still another kit of the invention includes (i) an agent selected from the agents of Table 1; and (ii) instructions for administering the agent and a second agent to a patient having a neurodegenerative disease, wherein the second agent is useful for treating a neurodegenerative disease.

Finally, another kit includes (i) an agent that is useful for treating a neurodegenerative disease; and (ii) instructions for administering this agent with an agent selected from the agents of Table 1 to a patient having a neurodegenerative disease.

The compositions, methods, and kits of the invention may be used to treat any neurodegenerative disease, including spinal muscular atrophy (SMA), spinal and bulbar muscular atrophy (SBMA), amyolateral sclerosis (ALS), Alzheimer's disease, Parkinson's diseases, and Huntington's disease.

The invention also features a method of identifying a combination that may be useful for the treatment of a neurodegenerative disease. This method includes the steps of: (a) contacting SMN-expressing cells with a combination comprising an agent selected from the agents of Table 1 and a candidate compound; and (b) determining whether the combination of the agent and the candidate compound increase the amount of SMN protein relative to cells contacted with the agent but not contacted with the candidate compound, wherein an increasing in the amount of SMN protein identifies the combination as a combination useful for the treatment of a neurodegenerative disease. Desirably, the cells are mammalian cells (e.g., human fibroblasts from an SMA patient)

The compositions, methods, and kits of the invention may be used to treat any neurodegenerative disease, including spinal muscular atrophy (SMA), spinal and bulbar muscular atrophy (SBMA), amyolateral sclerosis (ALS), Alzheimer's disease, Parkinson's diseases, and Huntington's disease.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

By “an amount sufficient” is meant the amount of a compound, alone or in combination with another therapeutic regimen, required to treat, prevent, or reduce a metabolic disorder such as diabetes in a clinically relevant manner. A sufficient amount of an active compound used to practice the present invention for therapeutic treatment of conditions caused by or contributing to diabetes varies depending upon the manner of administration, the age, body weight, and general health of the mammal or patient. Ultimately, the prescribers will decide the appropriate amount and dosage regimen. Additionally, an effective amount may be an amount of compound in the combination of the invention that is safe and efficacious in the treatment of a patient having a metabolic disorder such as diabetes over each agent alone as determined and approved by a regulatory authority (such as the U.S. Food and Drug Administration).

By “more effective” is meant that a treatment exhibits greater efficacy, or is less toxic, safer, more convenient, or less expensive than another treatment with which it is being compared. Efficacy may be measured by a skilled practitioner using any standard method that is appropriate for a given indication.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that reduces glucose levels and that is formulated for administration by inhalation will differ from a low dosage of the same agent formulated for oral administration.

By a “high dosage” is meant at least 5% (e.g., at least 10%, 20%, 50%, 100%, 200%, or even 300%) more than the highest standard recommended dosage of a particular compound for treatment of any human disease or condition.

By a “candidate compound” is meant a chemical, be it naturally-occurring or artificially-derived. Candidate compounds may include, for example, peptides, polypeptides, synthetic organic molecules, naturally occurring organic molecules, nucleic acid molecules, peptide nucleic acid molecules, and components and derivatives thereof.

Compounds useful in the invention include those described herein in any of their pharmaceutically acceptable forms, including isomers such as diastereomers and enantiomers, salts, esters, solvates, and polymorphs thereof, as well as racemic mixtures and pure isomers of the compounds described herein.

Other features and advantages of the invention will be apparent from the detailed description and from the claims.

### BRIEF DESCRIPTION OF THE DRAWINGS

FIGS. 1A and 1B are graphs showing SMN protein levels in patient (GM03813) versus carrier fibroblast cells (GM03814). Fibroblast cell lines were analyzed by SMN cytoblot, using anti-SMN antibody from BD Biosciences (FIG. 1A). A scatter plot of the data used is also shown (FIG. 1B).

FIG. 2 is a series of illustrations depicting absolute SMN fold induction of various drug combinations. SMN fold induction was calculated as SMN(T−B)/SMN(U−B) where “T” is the signal from treated cells, “B” is plate-specific background, and “U” is the signal from untreated cells.

FIG. 3 is a series of illustrations depicting viability-controlled fold induction of various drug combinations. Viability-controlled fold induction was calculated as (SMN fold induction)/(ATP fold induction), where ATP fold induction is calculated in the same manner as SMN fold induction, or: ATP(T−B)/ATP(U−B).

### DETAILED DESCRIPTION

We have identified agents that increase SMN protein levels in SMA fibroblasts in vitro. These agents may be used to increase SMN protein levels in patients having a neurodegenerative disease (e.g., SMA, SBMA, ALS, Alzheimer's disease, Parkinson's disease, or Huntington's disease), and may further be used to treat these patients.

TABLE 1
Agent

1-Boc-homopiperazine
2-(4-methyl010cyclohexylidene) acetic acid
2-N-Butylthiophene
3,4-Dimethoxyphenylacetone
4-Pentenoic acid
5-10-Dihydro-5-10-dimethylphenazine
Acetrizoate
Allyl Disulfide
alpha-Bromohepyanoic acid
Altretamine
Alosetron
Amantadine
Amikacin
Amrinone
Anisotropine Methylbromide
Ascorbic Acid
Azlocillin
Beclomethasone
Benfluorex
Benzyl Benzoate
Beta-Ionol
Betaxolol
Bethanechol Chloride
Bezafibrate
Bis(2-Ethlyhexyl) Fumarate
Boldine
Busulfan
Calcitonin
Calcium Chloride
Carbenicillin
Chlophedianol
Chlortetracycline
Chymopapain
Cinoxacin
Citronellal
Creatine
Cupric Chloride
Cyclosporine
Desoxycorticosterone Acetate
Dibekacin
Divalproex
dl-Penicillamine
Dobutamine
Efavirenz
Ellipticine
Enalapril
Epinephrine
Ethionamide
Ethopropazine
Fenpiverinium Bromide
Ferric Ammonium Citrate
Ferrous Sulfate
Fluocinonide
Fosfosal
Framycetin
Gabapentin
Gadoteridol
Gallamine Triethiodide
Gentamicin
Geranyl Acetate
Glutathione Disulfide
Guanethidine
Guanfacine
H-8
Hexamethyltetracosane
Indoprofen
Isopulegol acetate
Khellin
Lactitol
Levetiracetam
L-Methionine Sulfoximine
Loxapine
L-Tartaric acid
Manganese Sulfate
Mastic
Memantine
Menthol
Metampicillin
Methyl Linoleate
Methyl Oleate
Methyl Palmitoleate
Moxisylyte
Neomycin
Nifuroxazide
Nonoxynol-9
Oxaceprol
Oxprenolol
Pargyline
Paromomycin
Pazufloxacin
Pentagastrin
Pergolide
Piperazine
Propranolol
Pyrantel
Pyridostigmine Bromide
Rescinnamine
S-Allyl-L-Cysteine
Selenium Sulfide
Serotonin
Sirtinol
Spectinomycin
Tegafur
Teicoplanin
Tiapride
Trans-Anethole
Ubenimex
Ursodiol
Vincamine

A discussion of some of the agents listed in Table 1 now follows.

Guanidinium-Containing Compounds

Compounds that can be used in the compositions, methods, and kits of the invention include guanidinium-containing compounds such as guanfacine, guanethidine, creatine, guamecycline, guanabenz, guanadrel, guanoxabenz, and guanoxan. Guanfacine (N-aminoiminomethyl)-2,6-dichlorobenzeneacetamide) is an alpha adrenergic receptor agonist. It's chemical structure and methods of making it are described in U.S. Pat. No. 3,632,645. Guanethidine ([2-(hexahydro-1 (2H)-azocinyl)ethyl]guanidine) is an anti-hypertensive norepinephrine-depleting agent. It's chemical structure and methods of making it are described in U.S. Pat. No. 2,928,829. Analogs of any of the foregoing can also be used in the compositions, methods, and kits of the invention. Such analogs are described in U.S. Pat. Nos. 2,928,829; 3,247,221; 3,547,951; 3,591,636; 3,632,645; GB 1019120; and GB 1042207, each of which is hereby incorporated by reference.

Transition Metal Salts

Compounds that can be used in the compositions, methods, and kits of the invention include transition metal salts such as manganese salts, ferric and ferrous salts, and cupric salts. Exemplary transition metal salts are manganese sulfate, ferric ammonium citrate, ferrous sulfate, cupric sulfate, cupric chloride, and copper bis-3,5-diisopropylsalicylate. Each of these transition metal salts acts as an antioxidant and free radical scavenger. Other antioxidants and free radical scavengers may be used in the compositions, methods, and kits of the invention.

Analogs

Analogs of any of the compounds listed in Table 1 may be used in any of the methods, kits, and compositions of the invention. Analogs are known in the art (e.g., as described herein). Altretamine analogs are described in U.S. Pat. No. 3,424,752; alosetron analogs are described in U.S. Pat. No. 5,360,800; amikacin analogs are described in U.S. Pat. No. 3,781,268; amrinone analogs are described in U.S. Pat. Nos. 4,004,012 and 4,072,746; anisotropine methylbromide analogs are described in U.S. Pat. No. 2,962,499; azlocillin analogs are described in U.S. Pat. No. 3,933,795; beclomethasone analogs are described in U.S. Pat. No. 3,312,590 and Great Britain Patent Nos. 912378 and 901093; benfluorex analogs are described in U.S. Pat. No. 3,607,909; betaxolol analogs are described in U.S. Pat. No. 4,252,984; bethanechol chloride analogs are described in U.S. Pat. Nos. 1,894,162 and 2,322,375; bezafibrate analogs are described in U.S. Pat. No. 3,781,328; calcitonin analogs are described in German Patent No. 1929957; carbenicillin analogs are described in U.S. Pat. Nos. 3,142,673 and 3,282,926; chlophedianol analogs are described in U.S. Pat. No. 3,031,377; chlortetracycline analogs are described in U.S. Pat. Nos. 2,899,422, 2,987,449, and 3,050,446; chymopapain analogs are described in U.S. Pat. No. 3,558,433; cinoxacin analogs are described in U.S. Pat. No. 3,669,965; dibekacin analogs are described in German Patent No. 2135191; dobutarnine analogs are described in U.S. Pat. No. 3,987,200; efavirenz analogs are described in U.S. Pat. No. 5,519,021; ellipticine analogs are described in U.S. Pat. Nos. 3,933,827, 4,045,565, 4,310,667,4,434,290, 4,483,989, 4,698,423, and 4,851,417; enalapril analogs are described in U.S. Pat. No. 4,374,829; ethionamide analogs are described in Great Britain Patent No. 800250; ethopropazine analogs are described in U.S. Pat. No. 2,607,773; fenpiverinium bromide analogs are described in Great Britain Patent No. 708859; fosfosal analogs are described in German Patent No. 2641526; gabapentin analogs are described in U.S. Pat. No. 4,024,175; gadoteridol analogs are described in U.S. Pat. No. 4,885,365; gallamine triethiodide analogs are described in U.S. Pat. No. 2,544,076; gentarnicin analogs are described in U.S. Pat. Nos. 3,091,572 and 3,136,704; guanethidine analogs are described in U.S. Pat. No. 2,928,829; guanfacine analogs are described in U.S. Pat. No. 3,632,645; levetiracetam analogs are described in U.S. Pat. No. 4,943,639; loxapine analogs are described in U.S. Pat. No. 3,546,226; memantine analogs are described in U.S. Pat. No. 3,391,142; metampicillin analogs are described in Great Britain Patent No. 1081093; moxisylyte analogs are described in German Patent No. 905738; neomycin analogs are described in U.S. Pat. Nos. 2,848,365 and 3,108,996; nifuroxazide analogs are described in U.S. Pat. No. 3,290,213; oxaceprol analogs are described in U.S. Pat. No. 3,860,607; oxprenolol analogs are described in Great Britain Patent No. 1077603; pargyline analogs are described in U.S. Pat. No. 3,155,504; paromomycin analogs are described in U.S. Pat. No. 2,916,876; pazufloxacin analogs are described in U.S. Pat. No. 4,990,508; pentagastrin analogs are described in U.S. Pat. No. 3,896,103; pergolide analogs are described in U.S. Pat. No. 4,166,182; pyrantel analogs are described in U.S. Pat. No. 3,502,661; pyridostigmine bromide analogs are described in U.S. Pat. No. 2,572,579; rescinnamine analogs are described in U.S. Pat. Nos. 2,876,228 and 2,974,144; sirtinol ((2-[(2-hydroxy-naphthalen-1-ylmethylene)-amino]-N-(1-phenyl-ethy-1)-benzamide))analogs include (8,9-dihydroxy-6H-(1)benzofuro[3,2-c]chromen-6-one), M15 (1-[(4-methoxy-2-nitro-phenylimino)-methyl]-naphthalene-2-ol), butyrates (including sodium butyrate and sodium phenylbutyrate), tributytrin, trichostatin A (TSA), TPX-HA analog (CHAP compounds built from TSA and cyclic tripeptides, hydroxamic acid based), trapoxin, MS-275 (MS-27-275), NSC-706995, NSC-625748, NSC-656243, NSC-144168, psammaplin analogues, oxamflatin, apicidin and derivatives, chlamydocin analogues, dimethyl sulfoxide, depudecin, scriptaid, isoquinolineimide, depsipeptide (FR901228), N-acetyl dinaline, SAHA, suberic bis-hydroxamic acid, pyroxamide and analogues, m-carboxy cinnamic acid bis-hydroxamic acid (CBHA), cotara 131 1-chTNT-1/B, CI-944, valporate, splitomicin, allyl sulfur compounds, dimethylaminobenzamidylcaprylic hydroxamate (DBCH); and the compounds described in PCT Patent Publication WO 03/046207; spectinomycin analogs are described in U.S. Pat. Nos. 3,206,360, 3,234,092, and 3,272,706; tegafur analogs are described in Great Britain Patent No. 1168391; teicoplanin analogs are described in U.S. Pat. No. 4,239,751 and 4,542,018; tiapride analogs are described in Great Britain Patent No. 1394563; ubenimex analogs are described in U.S. Pat. Nos. 4,029,547 and 4,052,449; and vincamine analogs are described in U.S. Pat. No. 3,770,724.

Additional Therapeutic Regimens

If desired, the patient may also receive additional therapeutic regimens. For example, therapeutic agents may be administered with the agent or agents described herein at concentrations known to be effective or under investigational study for such therapeutic agents. Agents useful to treat a neurodegenerative disease include the following: compounds that correct aberrant SMN protein splicing or protein levels; calcium antagonists such as nimodipine; sodium channel blockers such as fosphenytoin, sipatrigine, and lubeluzole; caspase inhibitors such as p35, ZVAD, and crmiA; neuroimmunophilins; amino acids such as taurine and adenosine and other adenosine-based neuroprotectants; competitive and noncompetitive glutamate antagonists such as phencyclidine, ketamine, dizocilpine, dextromethorphan, magnesium, selfotel, MDL 104,653 (3-phenyl-4-hydroxy-7-chloroquinolin-2(1H)-one) and gavestinel; other agents that protect against glutamate-induced toxicity such as TRO 17416 (Trophos SA), TRO 19622 (Trophos SA), and the glutamate receptor agonist TCH-346 (dibenzo[b,f]oxepin-10-ylmethyl-prop-2-ynylamine); benzothiazole class members such as riluzole; free radical scavengers and agents that reduce nitric oxide-related toxicity such as NXY-059 (disodium 2,4-disulfophenyl-N-tert-butylnitrone), lipoic acid, quercitin, peroxynitrite, and lubeluzole; inhibitors of apoptosis such NAIP; growth and trophic factors such as nerve growth factor and glial cell line-derived neurotrophic factor; agents that lower intracellular calcium levels; GABAα receptor activators such as clomethiazole; inhibitors of Rho kinase such as BA-1016 (BioAxone Therapeutic Inc.); Rho antagonists such as Cethrin (BioAxone Therapeutic Inc.; U.S. Pat. No. 6,855,688); protein-based therapeutics such as RI-820; agents that stabilize the neuronal membrane potential; neurosteroids such as allopregnanolone and dehydroepiandrosterone; anti-inflammatory or analgesic agents such as non-steroidal anti-inflammatory agents; tetracycline compounds such as minocycline; neuropeptides such as neuropeptides (opioid peptides, thyreoliberine, neuropeptide Y, galanin, VIP/PACAP, hormones such as estrogen and progestin, and caffeine); Co-enzyme Q10; creatinine; hydroxyurea; sodium or phenyl butyrate or other butyrate compounds; HDAC inhibitors such as valproate or valproic acid; aclarubicin; gabapentin; albuterol; quinazolines; aminogylcosides; and salbutamol.

Other agents useful to treat a neurodegenerative disease are (−)-epigallocatechin-3-gallate; (R)-(−)-BPAP; 106362-32-7; remacemide; selegiline; 4-Cl-kynurenine; A-134974; A-366833; A-35380; A-72055; ABS-205; AC-184897; AC-90222; ACEA-1021 (licostinel); ADCI; AEG-3482; AGY-110; AGY-207; AK-275 (vasolex); alaptid; ALE-0540; AM-36; annovis; ampakines; amyloid-inhibiting peptides; AN-1792; andrographolide; APBPI-124; apoptosin; aptiganel; AR-139525; AR-15896 (lanicemine); AR-A-008055; donepezil; AR-R-17779; AR-R18565; ARRY-142886; ARX-2000; ARX-2001; ARX-2002; AS-600292; AS-004509; AS-601245; autovac; axokine; AZ-36041; BA-1016; Bay Q 3111 (BAY-X-9227; N-(2-ethoxyphenyl)-N′-(1,2,3-trimethylpropyl)-2-nitroethene-1,1-diamine); BD-1054; BGC-20-1178; BIMU-8 ((endo-N-8-methyl-8-azabicyclo-(3.2.1 )oct-3-yl)-2,3-dihydro-3-isopropyl-2-oxo-1H-benzimidazol-1-carboxamide); BLS-602; BLS-605; BMS-181100 (alpha-(4-fluorophenyl)-4-(5-fluoro-2-pyrimidinyl)-1-piperazine butanol); brasofensine; breflate; BTG-A derivatives; C60 fullerenes; CAS-493 (aloracetam); celecoxib; CEP-1347; CEP-3122; CEP-4143; CEP-4186; CEP-751; CERE-20; CGP-35348 (P-(3-aminopropyl)-P-diethoxymethylphosphinic acid); CHF-2060; CNIC-568; CNS-1044; CNS-2103; CNS-5065; coenzyme Q10; CP-132484 (1-(2-aminoethyl)-3-methyl-8,9-dihydropyrano(3,2-e)indole); CP-283097; CPC-304; CX-516; cyclophosphamide; cyclosporin A; dabelotine; DCG-IV (2-(2,3-dicarboxycyclopropyl)glycine); DD-20207; dehydroascorbic acid; dexanabinol; dexefaroxan; dihydroquinolines; diperdipine; dizocilpine; DMP-543; DP-103; DP-109; DP-b99; DPP-225; dykellic acid; E-2101; EAA-404 (midafotel); EAB-318; edaravone; EF-7412; EGIS-7444; EHT-202; eliprodil; emopamil; EP-475; EQA-00 (anapsos); ES-242-1; estrogen or estrogen/progesterone; ethanoanthracene derivatives; F-10981; F-2-CCG-I; FCE-29484A; FCE-29642A; FGF-9; FGF-16; ersofermin; formobactin; FPL-16283; GAG mimetics; galantamine derivatives; galdansetron; ganstigmine; gavestinel; GDNF (liatermine); GGF-2; GKE-841 (retigabine); glialines (throphix); GM-1 ganglioside; GP-14683; GPI-1337; GPI-1485; GR-73632; GR-89696 (methyl 4-((3,4-dichlorophenyl)acetyl)-3-(1-pyrrolidinylmethyl)-1-piperazinecarboxylate fumarate); GSK-3 inhibitors; GT-2342; GT-715; GV-2400; GYKI-52466 (4-(8-methyl-9H-1,3-dioxolo(4,5-h)(2,3)benzodiazepin-5-yl)-benzenamine); HBNF; HF-0220; HP-184 (N-(n-propyl)-3-fluoro-4-pyridinyl-1H-3-methylindol-1-amine hydrochloride); IAPs; IDN-6556; IGF modulators (e.g., neurocrine); igmesine; imidazole derivatives; imidazolyl nitrones; inosine; interferon alpha; interleukin-2-like growth factor; iometopane; ipenoxazone; itameline; KF-17329; KP-102 (alanyl-(2-naphthyl)alanyl-alanyl-tryptophyl-phenylalanyl-lysinamide); KRX-411; KW-6002 (istradefylline; 8-(2-(3,4-dimethoxyphenyl)ethenyl)-1,3-diethyl-3,7-dihydro-7-methyl-1H-purine-2,6-dione); L-687306 (3-(3-cyclopropyl-1,2,4-oxadiazol-5-yl)-1-azabicyclo(2.2.1 )heptane); L-687414; L-689560 (trans-2-carboxy-5,7-dichloro-4-(((phenylamino)carbonyl)amino)-1,2,3,4-tetrahydroquinoline); L-701252; lamotrigine; LAU-0501; lazabemide; leteprinim; LIGA-20; LY-178002 (5-((3,5-bis(1,1-dimethylethyl)-4-hydroxyphenyl)methylene)-4-thiazolidinone); LY-233536 (decahydro-6-(2H-tetrazol-5-ylmethyl)-3-isoquinolinecarboxylic acid); LY-235959 (decahydro-6-(phosphonomethyl)-3-isoquinolinecarboxylic acid); LY-274614; LY-302427; LY-354006; LY-354740 (2-aminobicyclo(3.1.0)hexane-2,6-dicarboxylic acid); LY-451395; MCC-257; MCI-225 (4-(2-fluorophenyl)-6-methyl-2-(1-piperazinyl)thieno(2,3-d)pyrimidine); MDL-100748 (4-((carboxymethyl)amino)-5,7-dichloroquinoline-2-carboxylic acid); MDL-101002; MDL-102288; MDL-105519; MDL-27266 (5-(4-chlorophenyl)-4-ethyl-2,4-dihydro-2-methyl-3H-1,2,4-triazol-3-one); MDL-28170 (carbobenzoxyvalylphenylalanine aldehyde); MDL-29951 (3-(4,6-dichloro-2-carboxyindol-3-yl)propionic acid); mecasermin; MEM-1003; mepindolol; metallotexa-phyrins; methylphenylethynylpyridine (MPEP); microalgal compound; milacemide; mirapex (pramipexole); MLN-519; MS-153; MT-5; N-3393; naltrindole derivatives; NAPVSIPQ; NBI-30702; NC-531; neotrofin; neramexane; nerve growth factor gene therapy; neublastin; neurocalc; neurostrol; NLA-715 (clomethiazole); NNC-07-0775; NNC-07-9202 (2,3-dioxo-6-nitro-7-sulfamoylbenzo(f)quinoxaline); noggin; norleu; NOX-700; NPS-1407; NPS-846; NRT-115; NS-1209; NS-1608 (N-(3-(trifluoromethyl)phenyl)-N′-(2-hydroxy-5-chlorophenyl)urea); NS-2330; NS-257; NS-377; NS-638 (2-amino-1-(4-chlorobenzyl)-5-trifluoromethylbenzimidazole); NS-649; NXD-5150; NXY-059; odapipam; olanzapine; ONO-2506; OPC-14117 (7-hydroxy-1-(4-(3-methoxyphenyl)-1-piperazinyl)acetylamino-2,2,4,6-tetramethylindan); P-58; P-9939; PACAP; palmidrol; PAN-811; pan-neurotrophin-1; PBT-1 (clioquinol); PD-132026; PD-150606 (3-(4-iodophenyl)-2-mercapto-(Z)-2-propenoic acid); PD-159265; PD-90780; PDC-008.004; PE21; phenserine; philanthotoxins; piperidine derivatives; PK-11195 (1-(2-chlorophenyl)-N-methyl-N-(1-methylpropyl)-3-isoquinolinecarboxamide); PN-277; PNU-101033E; PNU-157678; PNU-87663; POL-255; posatirelin; PPI-368; PRE-103; propentofylline; protirelin; PRS-211220; PYM-50028; QG-2283; rasagiline; REN-1654; REN-1820; RI-820; riluzole; RJR-1401; Ro-09-2210; rolipram; RPR-104632 (2H-1,2,4-benzothiadiazine-1-dioxide-3-carboxylate acid); RS-100642; S-14820; S-176251; S-34730-1; S-34730; S-18986; S-312-d (methyl 4,7-dihydro-3-isobutyl-6-methyl-4-(nitrophenyl)thieno(2,3-b)-pyridine-5-carboxylate); S-33113-1; sabeluzole; safinamide; SB-271046; SB-277011 (trans-N-(4-(2-(6-cyano-1,2,3,4-tetrahydroisoquinolin-2-yl)ethyl)cyclohexyl)-4-quinolinecarboxamide); SEMAX; SIB-1553A; SIB-1765F (5-ethynyl-3-(1-methyl-2-pyrrolidinyl)pyridine maleate); siclofen; SJA-6017 (N-(4-fluorophenylsulfonyl)-L-valyl-L-leucinal); SKF-74652; SL-34.0026; SLV-308; SNX-482; SP-(V5.2)C; SPC-9766; SPH-1371; SPM-914; SPM-935; SSR-180575; SSR-482073; sumanirole; SUN-C5174; survivins; SYM-2207; T-588 (1-(benzo(b)thiophen-5-yl)-2-(2-(N,N-diethylamino)ethoxy)ethanol hydrochloride); tacrine analogs (ABS-301, ABS-302, ABS-304); talampanel; taltirelin; TAN-950A (2-amino-3-(2,5-dihydro-5-oxo-4-isoxazolyl)propanoic acid); TC-2559; TCH-346; TGP-580; thurinex; TK-14; TP-20; traxoprodil; U-74500A (21-(4-(3,6-bis(diethylamino)-2-pyridinyl)-1-piperazinyl)-16-methylpregna-1,4,9(11 )triene-3,20-dione HC1); U-78517F (2-((4-(2,6-di-1-pyrrolidinyl-4-pyrimidinyl)-1-piperazinyl)methyl)-3,4-dihydro-2,5,7,8-tetramethyl-2H-1-benzopyran-6-ol.di-HCl); UK-351666; UK-356464; UK-356297; vanoxerine; VX-799; WAY-855; WIB-63480-2; WIN-67500; WIN-68100; WIN-69211; xaliprodene; YM-90K (6-(1H-imidazol-1-yl)-7-nitro-2,3(1 H,4H)-quinoxalinedione); ziconotide; and zonampanel.

If more than one agent is employed, therapeutic agents may be delivered separately or may be admixed into a single formulation. When agents are present in different pharmaceutical compositions, different routes of administration may be employed. Routes of administration for the various embodiments include, but are not limited to, topical, transdermal, and systemic administration (such as, intravenous, intramuscular, intrathecal, subcutaneous, inhalation, rectal, buccal, vaginal, intraperitoneal, intraarticular, ophthalmic or oral administration). As used herein, “systemic administration” refers to all nondermal routes of administration, and specifically excludes topical and transdermal routes of administration. Desirably, the agent of the invention and additional therapeutic agents are administered within at least 1, 2, 4, 6, 10, 12, 18, 24 hours, 3 days, 7 days, or 14 days apart. The dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds. Optionally, any of the agents of the combination may be administered in a low dosage or in a high dosage, each of which is defined herein.

The therapeutic agents of the invention may be admixed with additional active or inert ingredients, e.g., in conventional pharmaceutically acceptable carriers. A pharmaceutical carrier can be any compatible, non-toxic substance suitable for the administration of the compositions of the present invention to a mammal. Pharmaceutically acceptable carriers include, for example, water, saline, buffers and other compounds described for example in the Merck Index, Merck & Co., Rahway, N.J. Slow release formulation or a slow release apparatus may be also be used for continuous administration.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Dosages

Generally, when administered to a human, the dosage of any of the agents of the combination of the invention will depend on the nature of the agent, and can readily be determined by one skilled in the art. Typically, such dosage is normally about 0.001 mg to 2000 mg per day, desirably about 1 mg to 1000 mg per day, and more desirably about 5 mg to 500 mg per day. Dosages up to 200 mg per day may be necessary. Administration of each agent in the combination can, independently, be one to four times daily for one day to one year, and may even be for the life of the patient. Chronic, long-term administration will be indicated in many cases.

Additional Applications

If desired, the compounds of the invention may be employed in mechanistic assays to determine whether other combinations, or single agents, are as effective as the combination in treating neurodegenerative diseases (e.g., SMA) using assays generally known in the art, examples of which are described herein. For example, candidate compounds may be tested, alone or in combination (e.g., with an agent that is useful for treating a neurodegenerative disease, such as those described herein) and applied to fibroblasts derived from patients diagnosed as having SMA. After a suitable time, these cells are examined for SMN protein levels. An increase in SMN protein levels identifies a candidate compound or combination of agents as an effective agent to treat a neurodegenerative disease.

The agents of the invention are also useful tools in elucidating mechanistic information about the biological pathways involved in SMN protein regulation. Such information can lead to the development of new combinations or single agents for treating SMA or another neurodegenerative disease. Methods known in the art to determine biological pathways can be used to determine the pathway, or network of pathways affected by contacting cells (e.g., fibroblasts or motorneurons) with the compounds of the invention. Such methods can include, analyzing cellular constituents that are expressed or repressed after contact with the compounds of the invention as compared to untreated, positive or negative control compounds, and/or new single agents and combinations, or analyzing some other activity of the cell such as enzyme activity. Cellular components analyzed can include gene transcripts, and protein expression. Suitable methods can include standard biochemistry techniques, radiolabeling the compounds of the invention (e.g., ¹⁴C or ³H labeling), and observing the compounds binding to proteins, e.g. using 2D gels, gene expression profiling. Once identified, such compounds can be used in in vivo models (e.g., a mouse model for SMA) to further validate the tool or develop new agents or strategies to treat neurodegenerative diseases.

Application to Other Diseases

The agents listed in Table 1 may act by increasing transcription, modifying splicing, inducing translational read-through, and/or increasing protein stability, and thus may, alone or in combination, be useful for treating other diseases that are caused by low expression of a gene. Such diseases include cancers that can be sent into growth arrest by the up-regulation of tumor suppressor genes such as p53 and transcriptional targets of the retinoblastoma protein. Other diseases that may be treating by administration of one or more agents listed in Table 1 include diseases caused by low gene expression due to premature stop codons, such as Duchenne muscular dystrophy and cystic fibrosis. Diseases that arise from splicing defects include familial isolated growth hormone deficiency, type II (IGHD II), Frasier syndrome and other disorders that result from abnormal expression of the Wilms tumor suppressor gene (WT1), frontotemporal dementia and Parkinsonism linked to chromosome 17 (FTDP-17), Hutchinson-Gilford progeria syndrome (HGPS), myotonic dystrophy, retinitis pigmentosa, atypical cystic fibrosis, neurofibromatosis type I (NF1), Fanconi's anemia, and breast cancer susceptibility at the BRCA1/BRCA2 loci. Diseases that may benefit to therapies that increase protein stability include hematological malignancies and solid tumors, stroke, and ischemia.

Small Molecule Stimulators of SMN Protein

There are a variety of mechanisms that could lead to increases in SMN protein concentration; such mechanisms include transcription initiation and elongation, pre-mRNA splicing, mRNA decay and stability, translation initiation and elongation, and protein degradation. All of these mechanisms can be surveyed simultaneously by screening for small molecules that increase the amount of SMN protein in SMA patient fibroblasts. Subsequent to identifying compounds with this property, it will be possible to identify which of these specific mechanisms is responsible for each compound's effect.

We set out to identify single agents that increase the concentration of the survival motor neuron (SMN) protein in mammalian cells. The copy number of the human SMN2 gene, and by extension the amount of SMN protein, has been found to inversely correlate with SMA disease severity in both humans and mice. Thus, compounds that increase the amount of SMN protein in cells are likely to be effective therapies for patients with SMA.

### EXAMPLE 1

One method for monitoring SMN protein levels in cells is through use of a cytoblot assay, in which cells are fixed and probed with an antibody against a target protein of interest. We have used a cytoblot assay to determine the concentration of SMN protein in SMA patient fibroblasts, and have identified small molecules that increase SMN protein concentration.

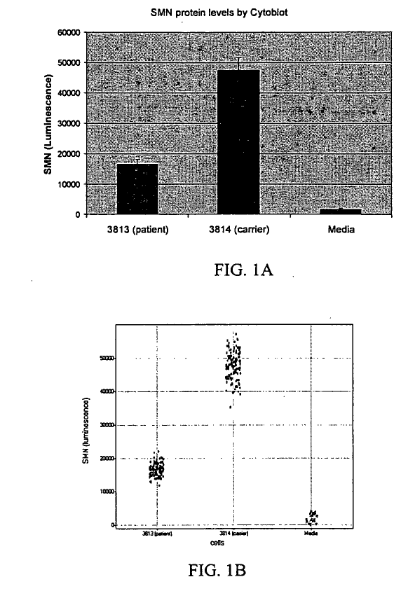

Using the cytoblot assay, we can clearly distinguish SMN protein levels in patient (GM03813) versus carrier fibroblast cells (GM03814), as shown in the FIGS. 1A and 1B. We performed parallel GAPDH cytoblot assays and verified that the difference in signal does not reflect a difference in cell number (data not shown). Parallel western blots show a similar distinction between the two fibroblast lines, and also demonstrate that the antibody used (from BD Biosciences) is highly specific for SMN protein.

For comparison with other high-throughput screening (HTS) assays, we have calculated a measure of signal to background referred to as the Z′ factor (26). We have looked at the for the distinction of patient versus carrier. For the samples above, where p is the patient (3813), and c is the carrier (3814), the Z′ factor was calculated as follows: Z′=1−(3*σ_{c}+3*σₚ)/(μ_{c}−μₚ). The data presented above correspond to a Z′ score of 0.45 when comparing patient (3813) to carrier (3814), and 0.61 for the patient to media control (note that this measures variability within a plate only). While this number cannot be directly linked to a probability, it is useful for comparing to other HTS assays. In our experience, these numbers fall within the range of a desirable HTS assay that can be used with a manageable number of replicates.

We also looked at a standard statistical comparison of two populations, the signal-to-noise ratio (SNR), which is similar to a student's t-test: SNR=(μ_{c}−μₚ)/(ν_{c}²+σₚ²)^{0.5}. Our data above show that the signals from the patient 3813 and carrier 3814 cell lines are separated by a SNR of 7.04, which corresponds to a confidence value of >>99% for distinction of the two cell lines. One can also look at the patient cell line alone and ask what level of increase we would expect to see with our assay. Because our standard deviation of the 3813 cell sample was about 0.1 times the average signal, we expect to be able to detect 3 standard deviations, or 30% changes, with 99% confidence.

The compounds identified as increasing SMN protein are listed in Table 1, above. Several of these are also listed in Table 2, below, along with their SNR.

TABLE 2

| Agent | Scoring | SNR |
|---|---|---|
| Ferric Ammonium | Visual | 19.4 |
| Citrate | | |
| Busulfan | Automated | 17.4 |
| Lactitol | Automated | 15.8 |
| Gallamine | Automated | 15.4 |
| Triethiodide | | |
| Gentamicin | Automated | 14.9 |
| Sulfate | | |
| Moxisylyte | Automated | 14.3 |
| Hydrochloride | | |
| Ethionamide | Automated | 14 |
| Framycetin | Visual | 13.3 |
| Efavirenz | Automated | 13.1 |
| Acetrizoate | Automated | 12.8 |
| Sodium | | |
| Cupric Chloride | Visual | 12.1 |
| Isopulegol acetate | Automated | 11.4 |
| Guanethidine | Visual | 11.3 |
| Monosulfate | | |
| Paromomycin | Automated | 11 |
| Sulfate | | |
| Spectinomycin | Automated | 11 |
| 4-Pentenoic acid | Automated | 10.8 |
| Oxaceprol | Automated | 10.6 |
| Tiapride | Automated | 10.5 |
| Chlortetracycline | Automated | 10.4 |
| Hydrochloride | | |
| Manganese | Visual | 10.3 |
| Sulfate | | |
| Beta-Ionol | Automated | 10.3 |
| Altretamine | Automated | 10.2 |
| Loxapine | Automated | 10.1 |
| Hydrochloride | | |
| Methyl Linoleate | Automated | 10.1 |
| Metampicillin | Automated | 9.95 |
| Teicoplanin | Visual | 9.72 |
| dl-Penicillamine | Automated | 9.63 |
| Methyl | Automated | 9.63 |
| Palmitoleate | | |
| Ethopropazine | Automated | 9.61 |
| Hydrochloride | | |
| H-8 | Visual | 9.52 |
| Benzyl Benzoate | Automated | 9.42 |
| Bethanechol | Automated | 9.26 |
| Chloride | | |
| Ursodiol | Automated | 9.2 |
| Allyl Disulfide | Automated | 9.08 |
| S-Allyl-L- | Automated | 9.02 |
| Cysteine | | |
| 2-n- | Automated | 8.96 |
| Butylthiophene | | |
| Citronellal | Automated | 8.6 |
| alpha- | Automated | 8.41 |
| Bromohepyanoic | | |
| acid | | |
| Trans-Anethole | Automated | 8.35 |
| Creatine | Visual | 8.34 |
| Epinephrine | Automated | 8.26 |
| Bitartrate | | |
| Carbenicillin | Automated | 7.99 |
| Disodium | | |
| Neomycin Sulfate | Automated | 7.96 |
| Methyl Oleate | Automated | 7.89 |
| Menthol | Automated | 7.87 |
| Pazufloxacin | Automated | 7.83 |
| Levetiracetam | Automated | 7.74 |
| Amikacin Sulfate | Automated | 7.7 |
| Pergolide | Automated | 7.69 |
| Mesylate | | |
| Calcium Chloride | Automated | 7.63 |
| Ferrous Sulfate | Visual | 7.55 |
| Fenpiverinium Bromide | | 7.49 |
| Calcitonin | | 7.46 |
| Chlophedianol Hydrochloride | | 7.44 |
| Desoxycorticosterone | Automated | 7.41 |
| Acetate | | |
| Propranolol Hydrochloride | Automated | 7.38 |
| Bezafibrate | Automated | 7.38 |
| Bis(2-Ethlyhexyl) Fumarate | Automated | 7.33 |
| Cyclosporine | Automated | 7.3 |
| L-Methionine Sulfoximine | Automated | 7.29 |
| 3,4-Dimethoxyphenylacetone | Automated | 7.25 |
| Guanfacine Hydrochloride | Visual | 7.24 |
| Betaxolol Hydrochloride | Automated | 7.21 |
| Benfluorex Hydrochloride | Visual | 7.11 |
| Selenium Sulfide | Automated | 7.08 |
| Fluocinonide | Automated | 7.01 |
| Nonoxynol-9 | Automated | 7 |
| Nifuroxazide | Automated | 6.96 |
| Vincamine | Automated | 6.94 |
| L-Tartaric acid | Automated | 6.92 |
| Gabapentin | Automated | 6.84 |
| Chymopapain | Automated | 6.83 |
| Dibekacin Sulfate Salt | Automated | 6.79 |
| Fosfosal | Automated | 6.77 |
| Enalapril | Automated | 6.77 |
| Tegafur | Automated | 6.74 |
| Khellin | Automated | 6.73 |
| Geranyl Acetate | Automated | 6.71 |
| Azlocillin Sodium | Automated | 6.7 |
| Piperazine Citrate | Automated | 6.68 |
| 1-Boc-homopiperazine | Automated | 6.66 |
| Hexamethyltetracosane | Automated | 6.64 |
| Pentagastrin | Automated | 6.63 |
| Mastic | Automated | 6.62 |
| Divalproex Sodium | Automated | 6.59 |
| Gadoteridol | Automated | 6.54 |
| Oxprenolol Hydrochloride | Automated | 6.52 |
| Pargyline | Automated | 6.52 |
| Glutathione Disulfide | Visual | 6.49 |
| Dobutamine Hydrochloride | Automated | 6.47 |
| Anisotropine Methylbromide | Automated | 6.43 |
| Rescinnamine | Automated | 6.3 |
| Ascorbic Acid | Visual | 6.29 |
| Beclomethasone | Automated | 6.27 |
| Dipropionate | | |
| Pyrantel Pamoate | Automated | 6.22 |
| Pyridostigmine Bromide | Automated | 6.19 |
| 2-(4- | Automated | 6.16 |
| methyl010cyclohexylidene) | | |
| acetic acid | | |
| Serotonin | Automated | 6.15 |
| Cinoxacin | Automated | 6 |

SMN Cytoblot Protocol

The SMN cytoblot protocol is described below.

Day 1

1. Plate 50 μl per well of 300,000 cells/ml (15,000 cells per well) into Nunc white 384-well opaque-bottomed plates.
2. Incubate plates at 37° C., 5% CO₂ overnight.
   Day 2
1. Aspirate media from wells using Tecan plate washer.
2. Fix cells by adding 20 μl/well of cold methanol (kept in −20° C. freezer).
3. Incubate plates in 4° C. refrigerator for 10 minutes.
4. Aspirate methanol using Tecan plate washer.
5. Add 50 μl/well of primary Ab solution, (BD anti-SMN Ab diluted to 125 ng/ml in 10% fetal goat serum PBST), using multi-drop.
6. Seal plates and incubate overnight at room temperature.
   Day 3
1. After 16-20 hrs incubation, wash plates with PBST 2× using Tecan plate washer.
2. Add 50 μl of secondary Ab solution, (Santa Cruz goat anti-Mouse-HRP diluted to 67 ng/ml in 10% fetal goat serum PBST).
3. Seal plates and incubate 2 hrs at room temperature.
4. Wash plates with PBST 2× using Tecan plate washer.
5. Add 50 μl/well 10% fetal goat serum PBST.
6. Incubate in 4° C. refrigerator for 10 minutes.
7. Aspirate plates using plate washer and add ECL luminescence solution, 20 μl/well.
8. Measure luminescence on plate reader.
   Automated Scoring

We scored each compound based on the maximum signal-to-noise ratio (SNR) found among its dose curves. These scores are based on edge-corrected SMN data, using only plates that passed quality control. The untreated level on each plate was found by taking the median of the untreated wells, and for each treated well we calculated a log(T/U) ratio. Each curve was generated in triplicate on each plate, so each point on a compound's dose curve was obtained by determining the median for the replicate points. Each ratio thus has an error estimate from the scatter between the triplicate data points (1.5 times the median absolute deviation from the median). For each dose curve, we calculated the SNR for each data point log(T/U)/error, and chose the maximum point as our indicator of SMN induction activity. Since this is a signal-to-noise score, scores much greater than one indicate significant activity, assuming normal statistics. We decided to use a cutoff signal-to-noise ratio of 6. This selection represents a considerable enrichment towards visually-selected compounds. We identified 100 out of 2000 compounds producing scores >6. This included 13 of the 28 visual selections.

SMN Assay Visual Selection Criteria

1. Type 1: Raw Hits
(a) Increased SMN signal in multiple wells
(b) Consistency between replicate wells
(c) Signal looks significant relative to surrounding wells and overall quality of plate
(d) Signal not created by edge correction
(e) Additional weight given to hits that replicate in retest
(f) Plates that did not pass quality control may have been used as additional reinforcement if the signal was very strong relative to the noise

2. Type 2: Normalized Hits
(a) SMN signal flat or slightly up, while Alamar blue data indicates toxicity
(b) Additional weight given to hits that replicate in retest
(c) Plates that did not pass quality control may have been used as additional reinforcement if the signal was very strong relative to the noise

3. Type 3 Reversal of Curve Hits

### EXAMPLE 2

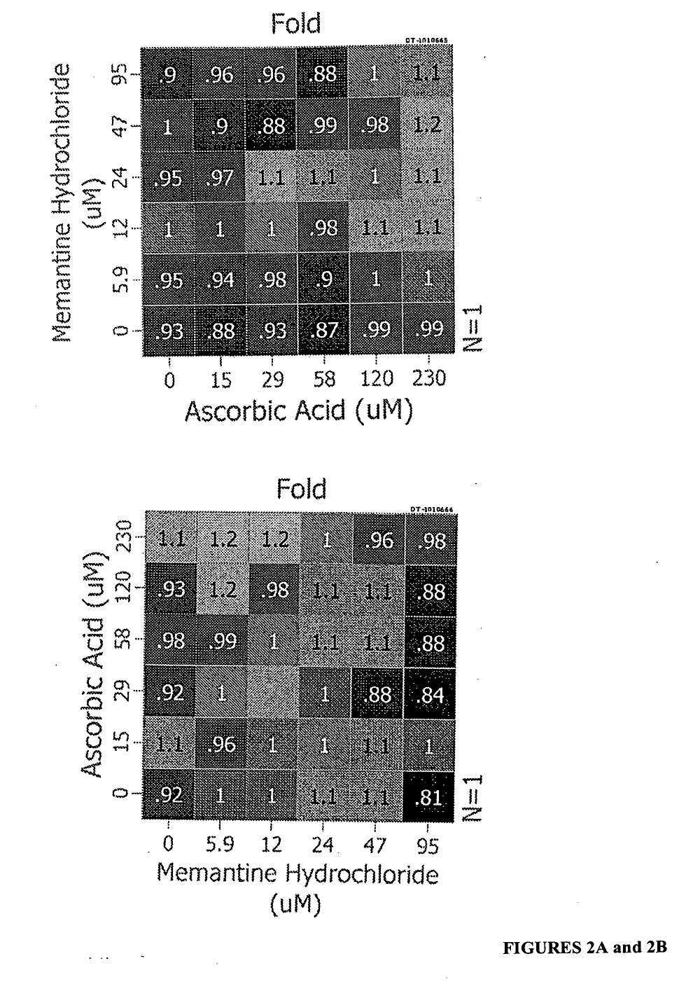

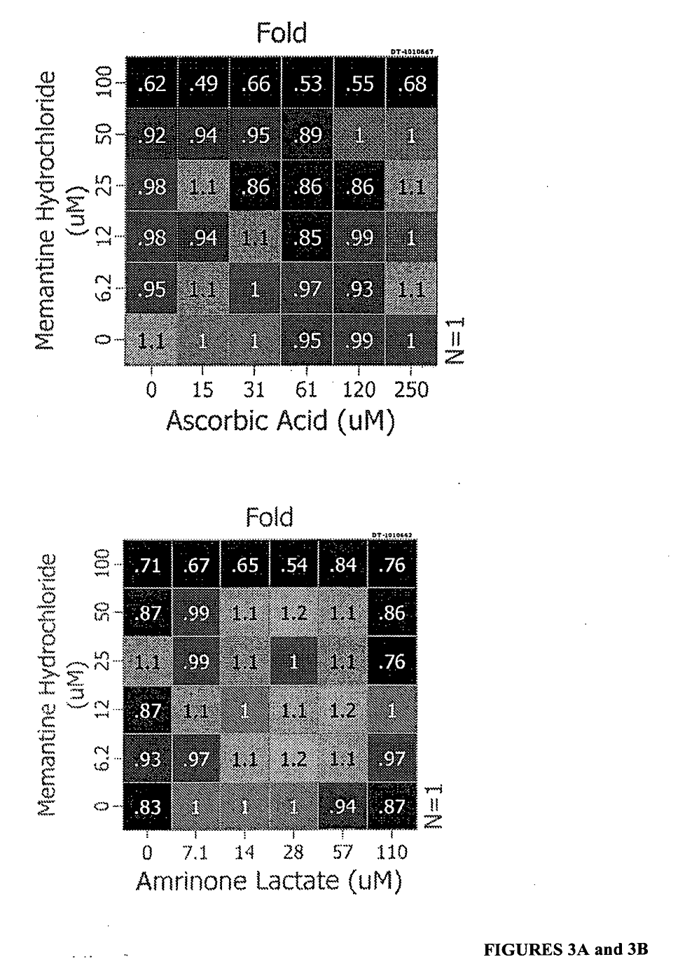

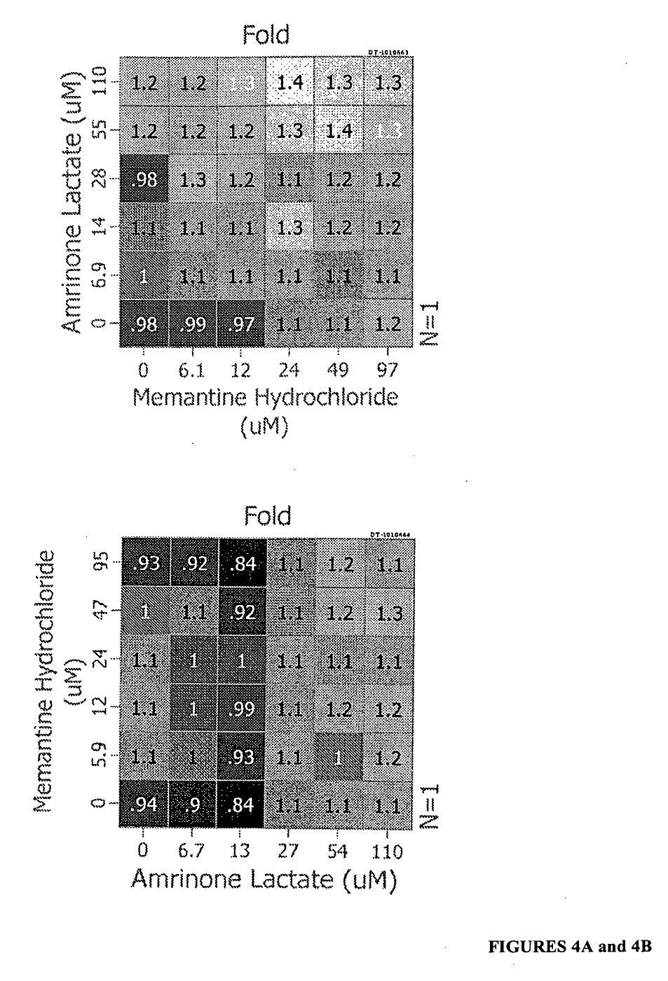

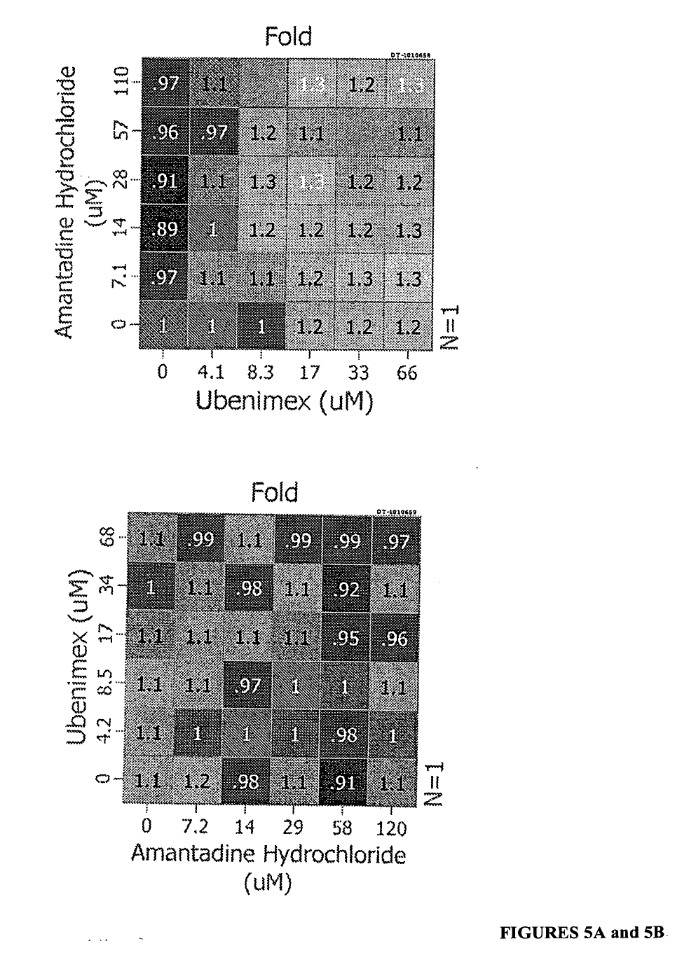

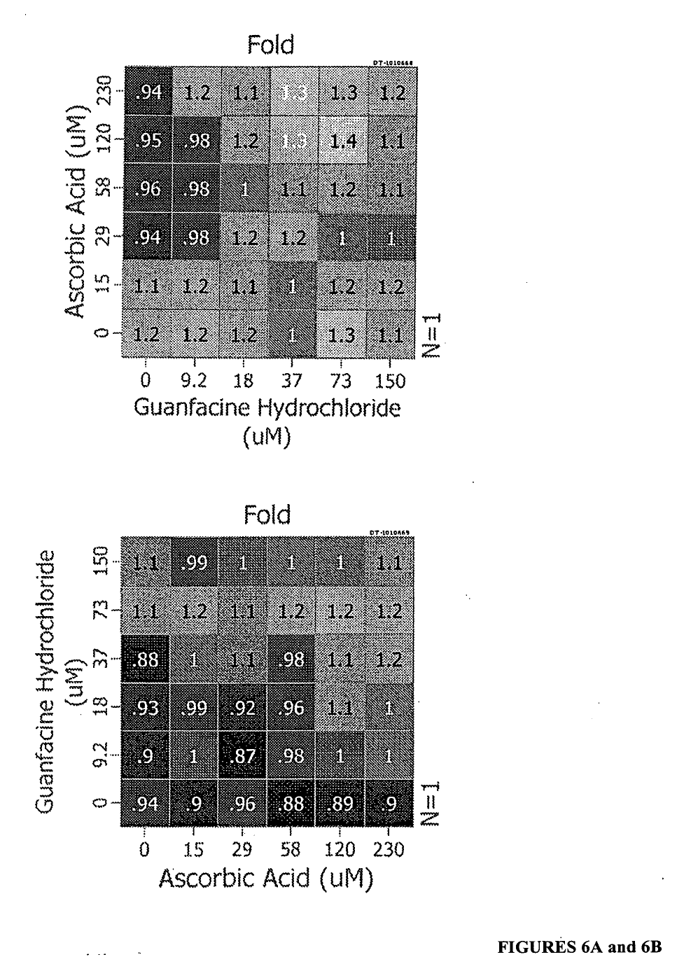

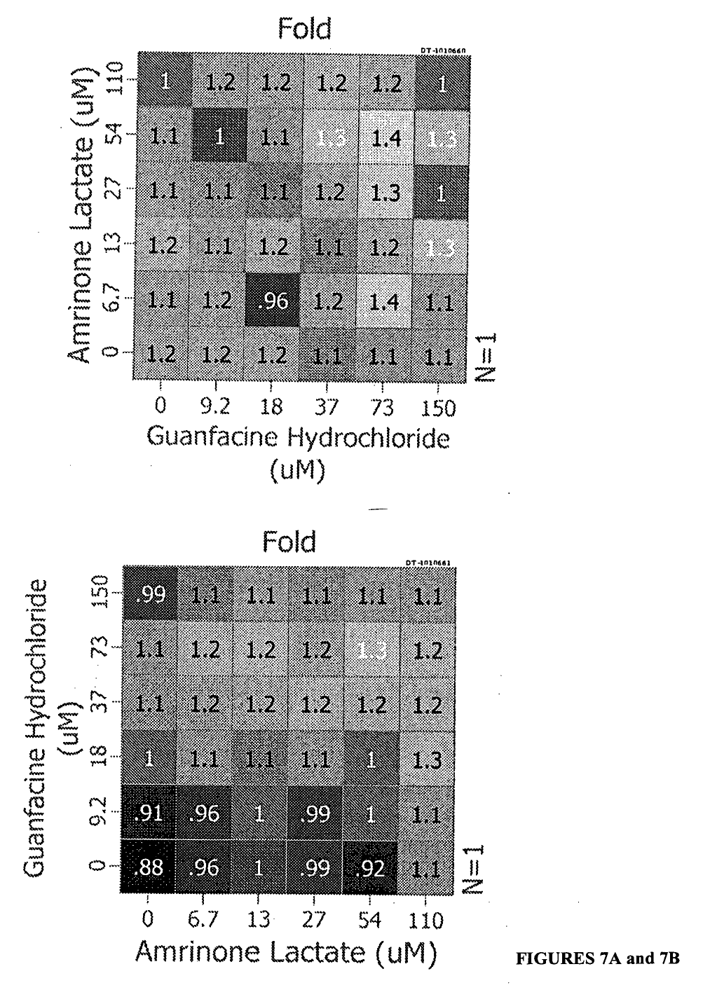

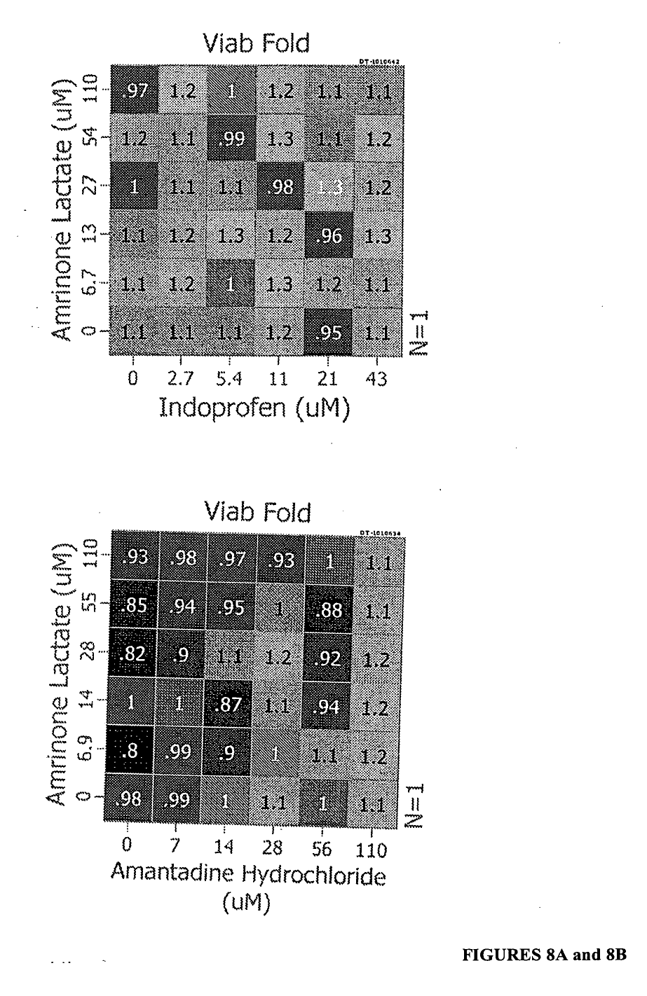

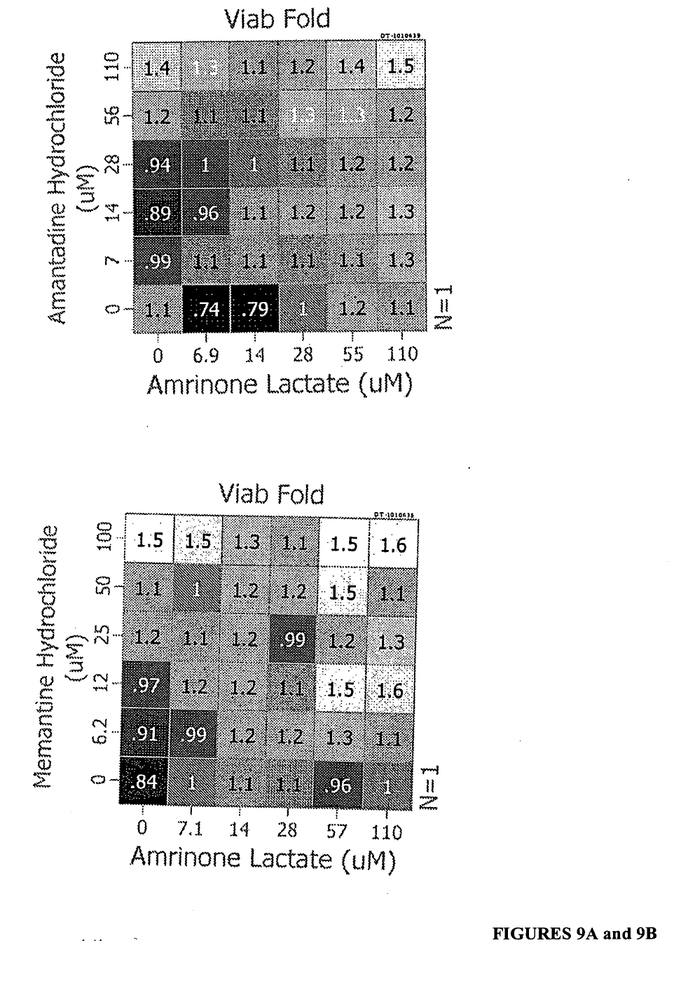

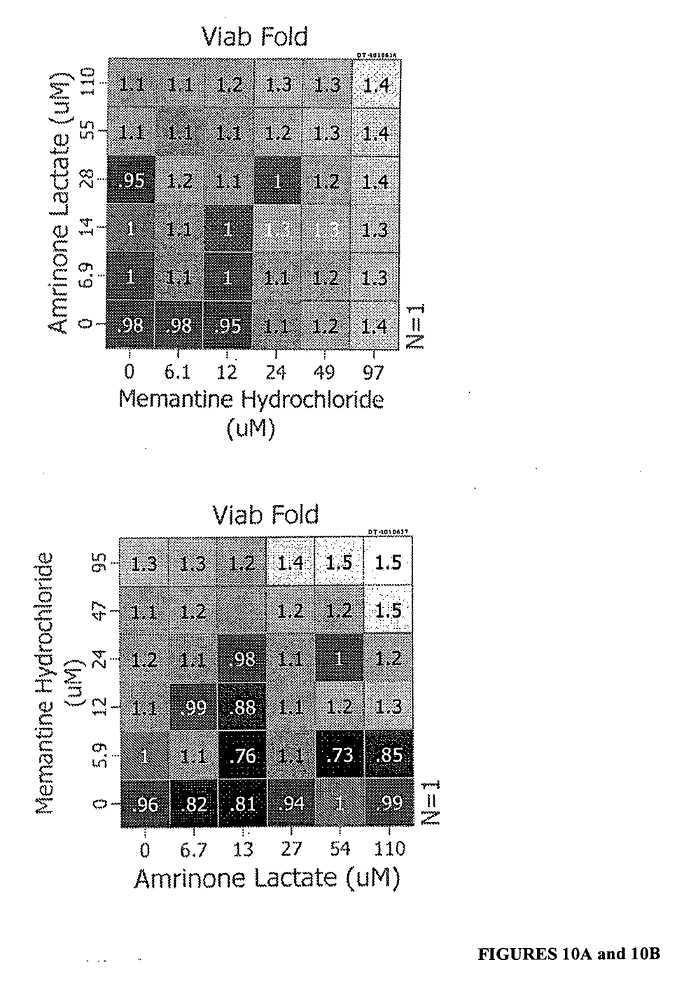

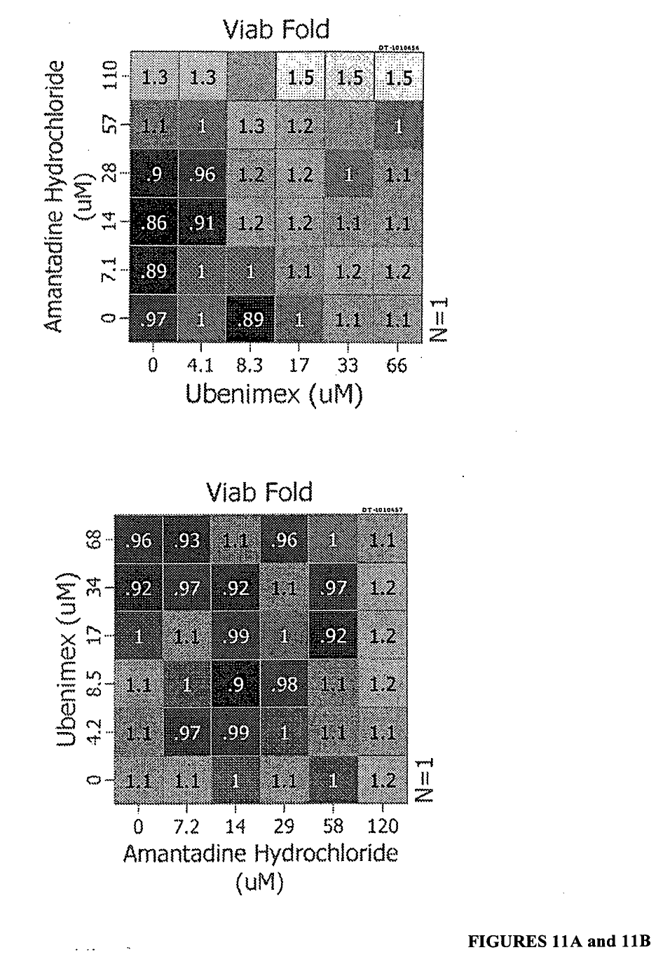

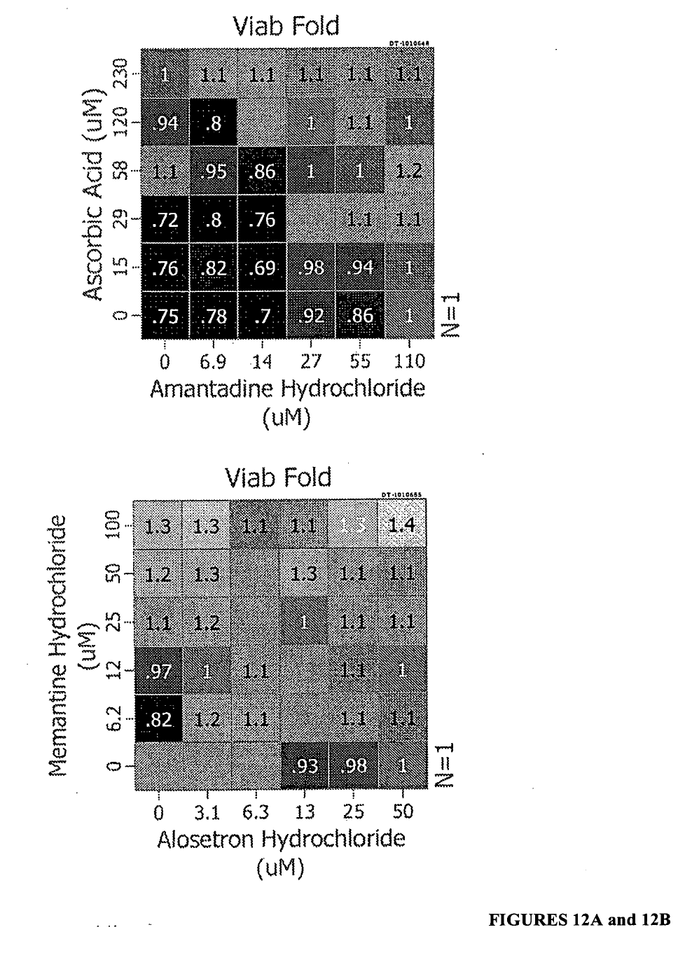

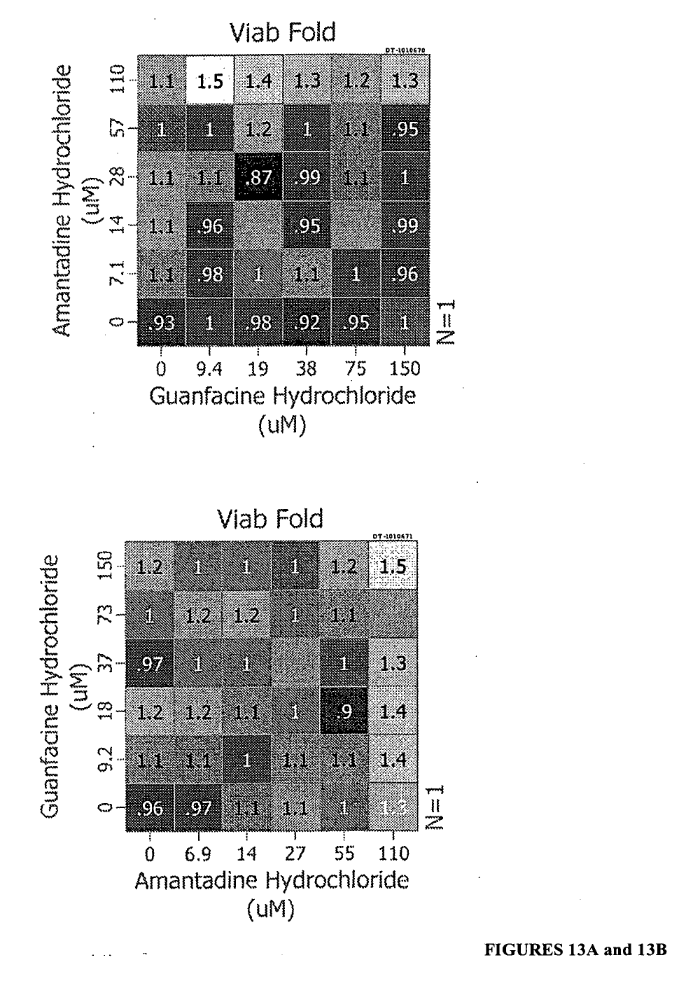

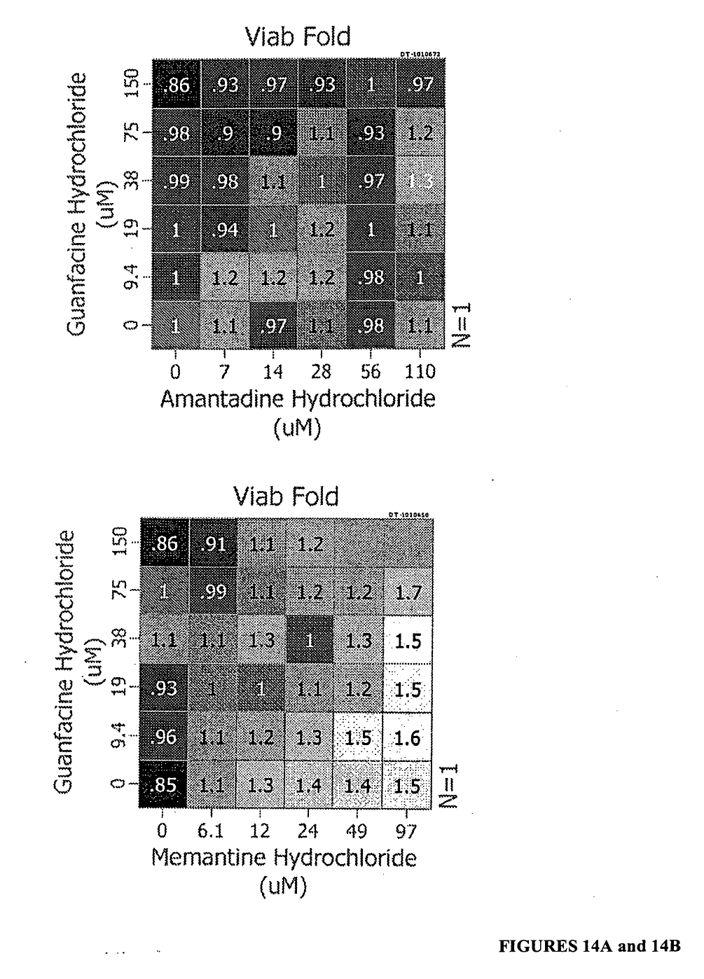

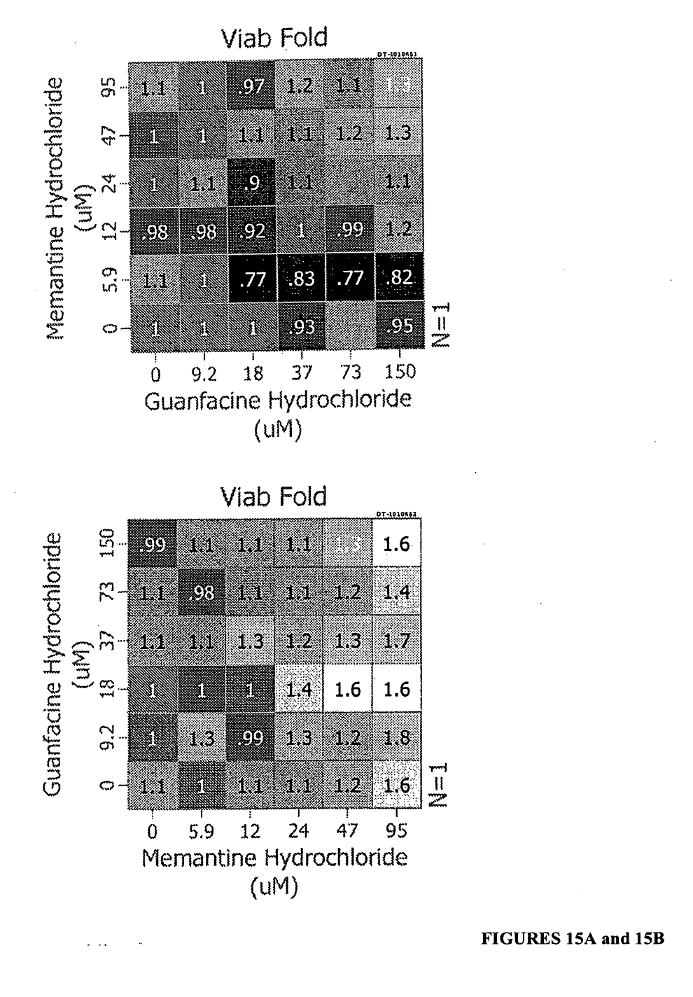

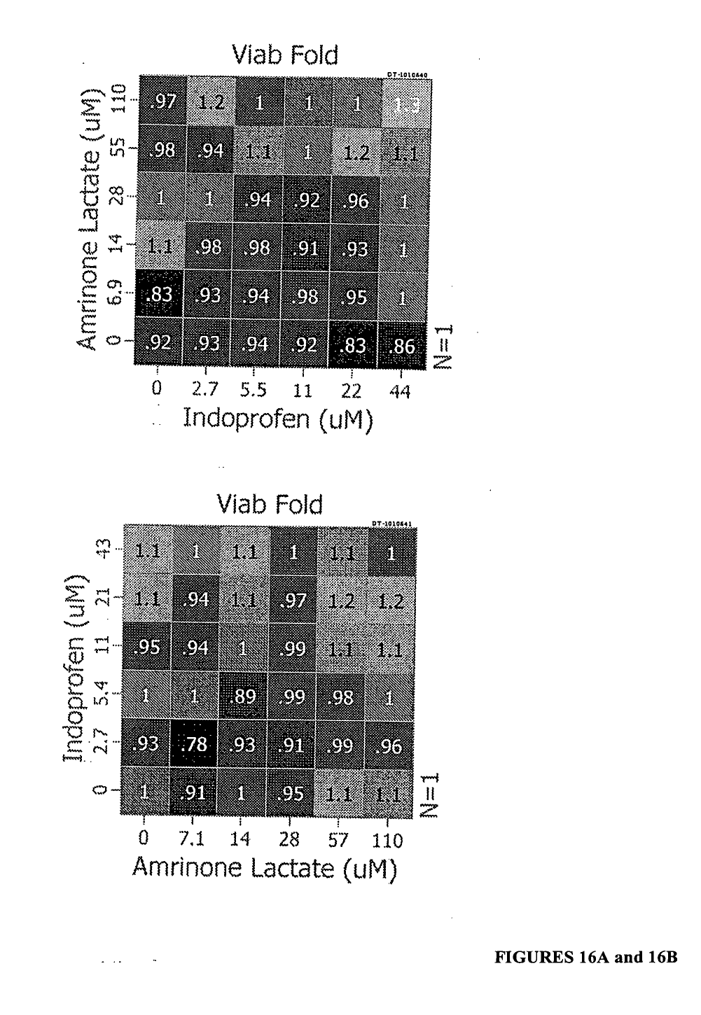

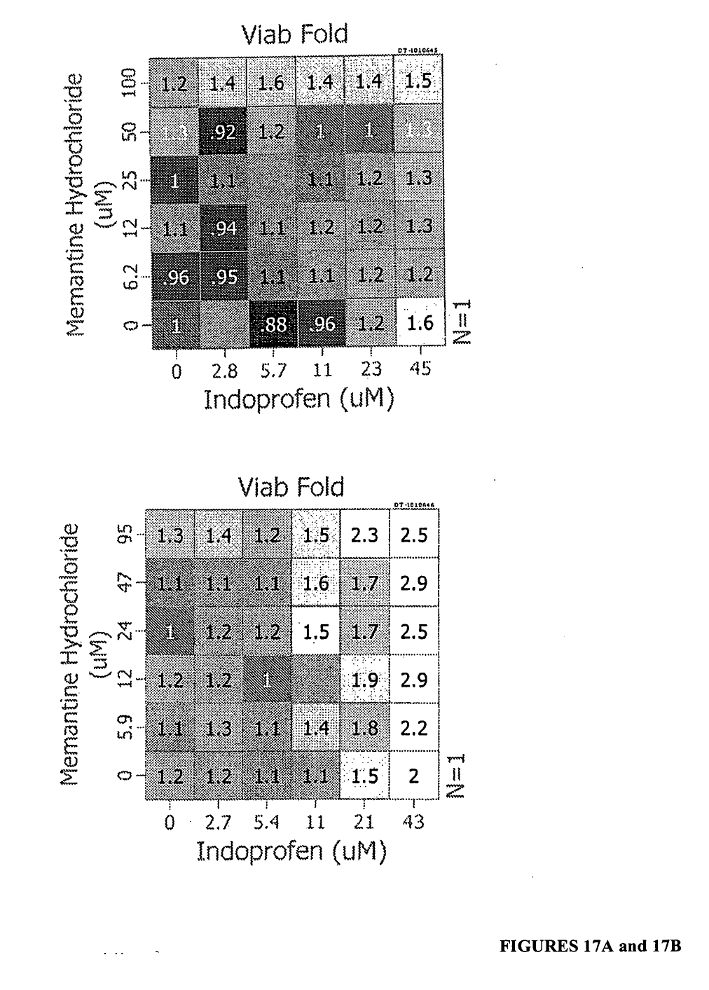

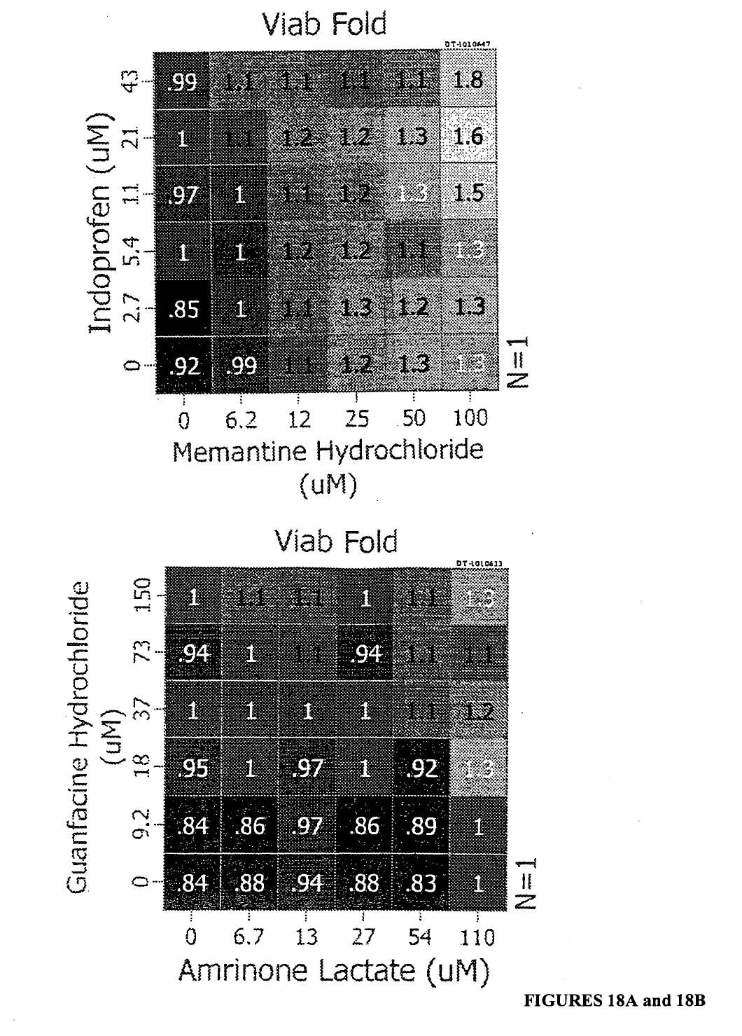

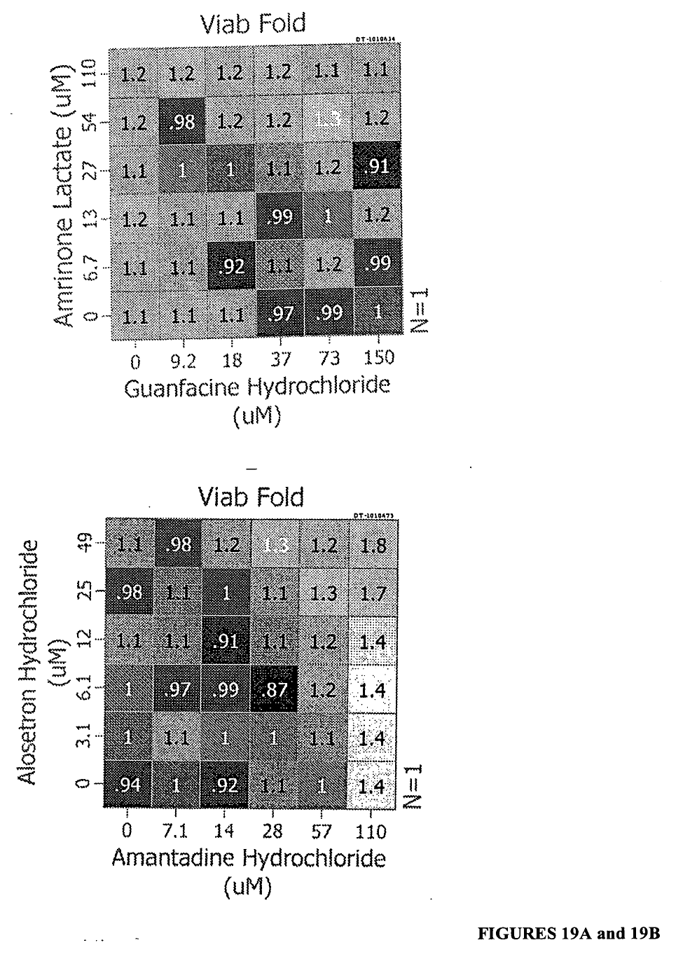

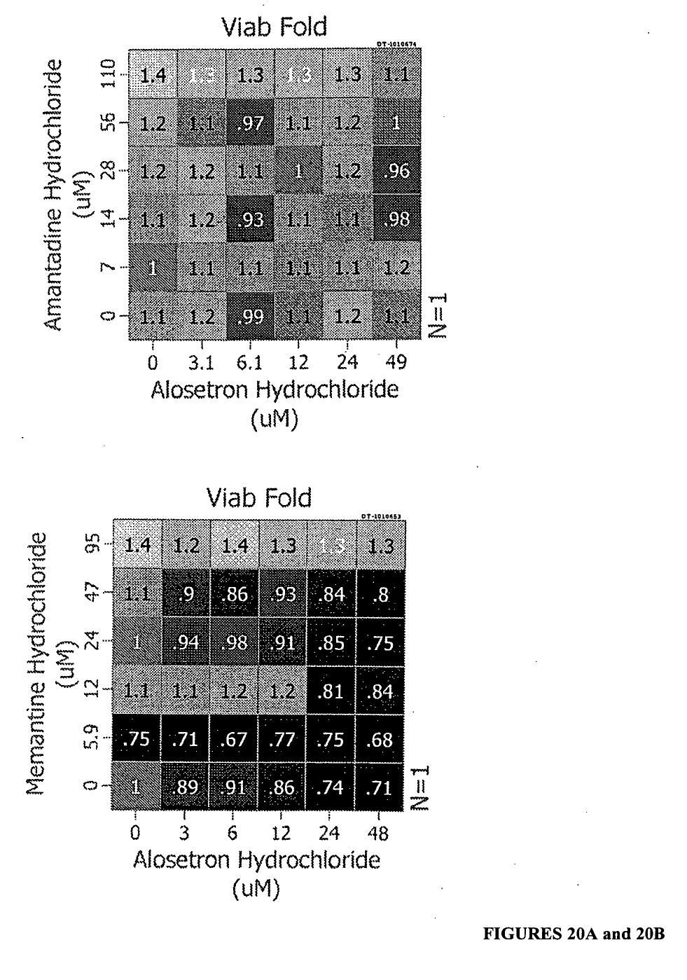

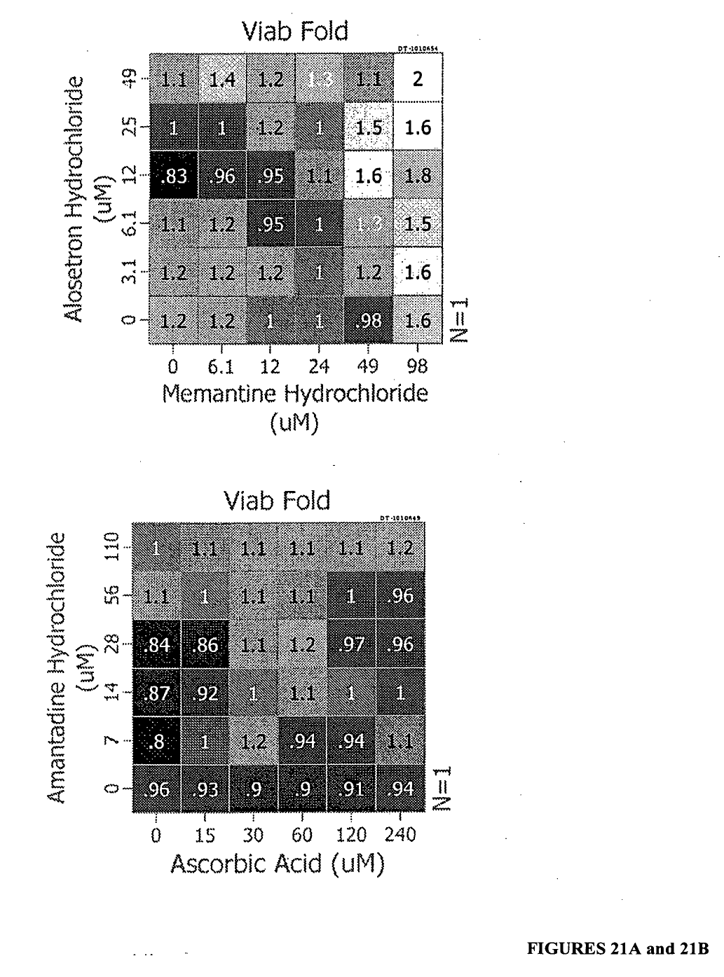

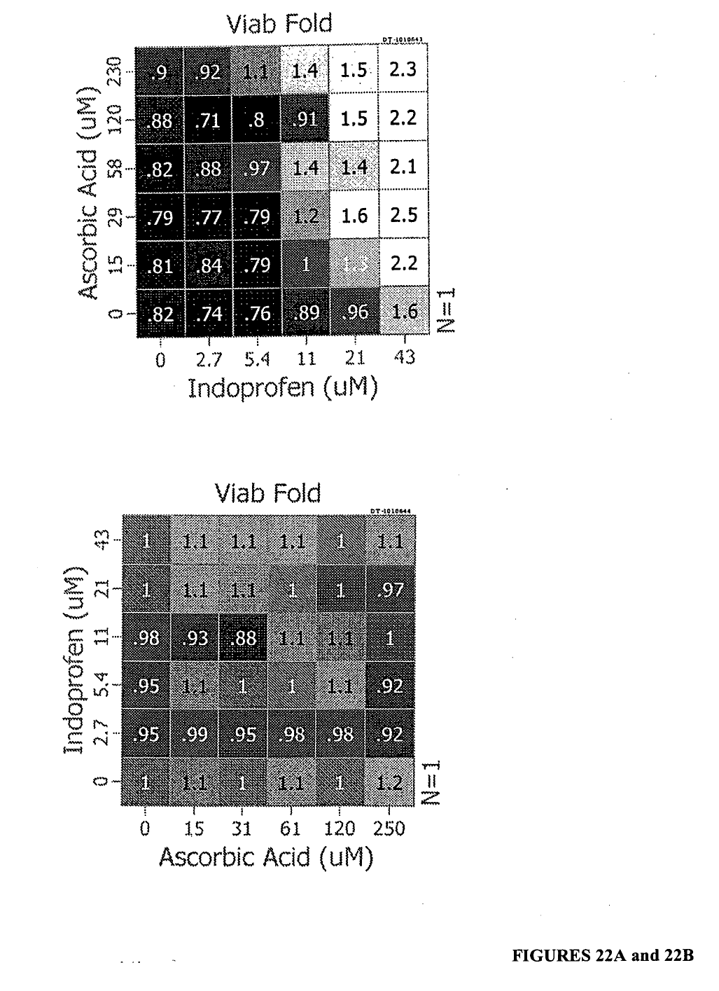

The following combinations were assayed to determine their ability to increase levels of SMN protein in GM03813 fibroblast cells: In certain embodiments, the two agents are ascorbic acid and memantine; ascorbic acid and indoprofen; ascorbic acid and amantadine; ascorbic acid and guanfacine; ubenimex and amantadine; amrinone and memantine; amrinone and amantadine; amrinone and indoprofen; amrinone and guanfacine; guanfacine and memantine; gunafacine and amantadine; alosetron and memantine; alosetron and amantadine; and indoprofen and memantine. The results are shown in FIGS. 2 and 3.

Methods

Day 1

Trypsinize confluent GM03813 fibrobast cells (passage 3-10) from Corning T-175 Tissue Culture flasks. Dilute cells to 89,000 cells/ml in MEM. Using multi-drop, add 45 μl/well to white 384-well opaque bottomed tissue culture treated plates. Incubate plates at 37° C., 5% CO₂ overnight.

Using PlateMate, add 60 μl per well to clear 384-well plates; one plate per master plate to be used.

Using “Two Drug MxM” program, transfer 3 μl from master to dilution plate (20× dilution) and 5 μl from dilution to assay plate (10× dilution) for a total 200× dilution of compounds. Create two daughter assay plates per combination of X and Y master plate. Spin plates briefly (˜30 seconds at 1000 RPM). Return assay plates to incubator for 72 hr incubation.

Day 4

ATP lite 1-step Addition: Reconstitute powder with assay buffer according to product instructions. Using PlateMate, add 50 μl per well to appropriate assay plates. Protect plates from light for ten minutes and place plates on orbital plate shaker (at least 700 RPM) for two minutes. Read plates on Wallac readers using SMAF_Lumi protocol.

Cell Fixation and Primary Antibody Addition: Remove remaining plates from incubator. Wash plates 2× using Tecan Plate washer with PBS, 0.1% Tween 20. Using PlateMate, add cold methanol (stored in −20° C. freezer) to plates, 30 μl/well. Incubate plates in 4° C. refrigerator for ten minutes. Repeat 2× washing using Tecans. Using PlateMate, add anti-SMN or antibody to plates, 40 μl/well. Seal plates and incubate at room temperature overnight.

Day 5

Secondary Antibody Addition and Luminescence: Wash plates 2× as above. Using PlateMate, add secondary antibody solution to plates, 30 μl/well. Seal plates and incubate at room temperature for two hours. After incubation wash plates 4× as above. Using PlateMate, add streptavidin-HRP solution to plates, 30 μl per well. Incubate 1.5 hours. Wash plates 3× as above. Using PlateMate, add Amersham ECL solution to plates, 20 μl/well. After ECL addition and before plate reading, dark adapt plates for approximately 3 minutes in order to eliminate luminescent signal from the plate itself. Measure luminescence on Wallac.

Data Analysis

Combination data were scored both as absolute SMN fold induction (FIG. 2) and as “viability controlled” SMN fold induction (SMN/ATP) (FIG. 3). SMN fold induction was calculated as SMN(T−B)/SMN(U−B) where “T” is the signal from treated cells, “B” is plate-specific background, and “U” is the signal from untreated cells. Viability controlled fold induction was calculated as (SMN fold induction)/(ATP fold induction), where ATP fold induction is calculated in the same manner as SMN fold induction, or: ATP(T−B)/ATP(U−B).

Combination data matrices were compared to the highest single agent (HSA) and Loewe additivity (ADD) models. HSA volume (HSA vol) and additivity volume (ADD vol) scores were determined for each matrix. The volume score is the sum across the entire matrix of the excess of the observed signal compared to the signal predicted by the model. Thus a score of 1 indicates that the matrix performs at the level predicted by the model.

### Other Embodiments

All publications, patent applications, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

What is claimed is:

1. A composition comprising:
(a) a first agent selected from the agents of Table 1; and
(b) a second agent useful for treating a neurodegenerative disease, wherein said first agent and said second agent are present in amounts that, when administered to a patient in need thereof, are sufficient to treat a neurodegenerative disease.

2. The composition of claim 1, further comprising one or more additional agents selected from Table 1.

3. The composition of claim 1, wherein said neurodegenerative disease is spinal muscular atrophy (SMA), spinal and bulbar muscular atrophy (SBMA), or amyolateral sclerosis (ALS).

4. The composition of claim 1, wherein said composition is formulated for oral administration.

5. The composition of claim 1, wherein said composition is formulated for systemic administration.

6. A method for treating a neurodegenerative disease, said method comprising administering to a patient in need thereof one or more agents selected from the agents of Table 1 in an amount sufficient to treat said neurodegenerative disease.

7. A method for treating a neurodegenerative disease, said method comprising administering to a patient in need thereof at least two different agents selected from the agents Table 1, wherein the first and second agents are administered simultaneously or within 28 days of each other, in amounts that together are sufficient to treat said neurodegenerative disease.

8. The method of claim 7, wherein said first and second agents are administered within 14 days of each other.

9. The method of claim 8, wherein said first and second agents are administered within 7 days of each other.

10. The method of claim 9, wherein said first and second agents are administered within 24 hours of each other.

11. The method of claim 6, wherein said neurodegenerative disease is SMA, SBMA, or ALS.

12. The method of claim 6, further comprising administering to said patient an additional agent useful for treating a neurodegenerative disease, wherein the agent or agents from Table 1 and said additional therapeutic agents are present in amounts that, when administered to said patient, are sufficient to treat said neurodegenerative disease.

13. The method of claim 6, wherein said agent or agents from Table 1 and said additional agent are administered within 14 days of each other.

14. The method of claim 13, wherein said agent or agents from Table 1 and said additional agent are administered within 7 days of each other.

15. The method of claim 14, wherein said agent or agents from Table 1 and said additional agent are administered within 1 day of each other.

16. The method of claim 6, wherein said agent or agents from Table 1 and/or said additional agent are administered orally or systemically.

17. The method of claim 6, wherein said neurodegenerative disease is SMA, SBMA, or ALS.

18. A kit comprising:
(i) an agent selected from the agents of Table 1; and
(ii) instructions for administering said agent to a patient having a neurodegenerative disease.

19. A kit comprising:
(i) a composition comprising two agents selected from the agents of Table 1; and
(ii) instructions for administering said composition to a patient having a neurodegenerative disease.

20. A kit comprising:
(i) a first agent selected from the agents of Table 1;
(ii) a second agent selected from the agents of Table 1; and
(iii) instructions for administering said first and said second agents to a patient having a neurodegenerative disease.

21. A kit comprising:
(i) an agent selected from the agents of Table 1; and
(ii) instructions for administering said agent with a second agent selected from the agents of Table 1 to a patient having a neurodegenerative disease.

22. A kit, comprising:
(i) a composition comprising
(a) a first agent selected from the agents of Table 1; and
(b) a second agent useful for treating a neurodegenerative disease; and
(ii) instructions for administering said composition to a patient having a neurodegenerative disease.

23. A kit, comprising:
(i) a first agent selected from the agents of Table 1; and
(ii) a second agent useful for treating a neurodegenerative disease; and
(iii) instructions for administering said first and said second agents to a patient having a neurodegenerative disease.

24. A kit comprising:
(i) an agent selected from the agents of Table 1; and
(ii) instructions for administering said agent and a second agent to a patient having a neurodegenerative disease, wherein said second agent is useful for treating a neurodegenerative disease.

25. A kit comprising:
(i) an agent useful for treating a neurodegenerative disease; and
(ii) instructions for administering said with an agent selected from the agents of Table 1 to a patient having a neurodegenerative disease.

26. A method of identifying a combination that may be useful for the treatment of a neurodegenerative disease, said method comprising the steps of:
(a) contacting cells with a combination comprising an agent selected from the agents of Table 1 and a candidate compound; and
(b) determining whether the combination of said agent and said candidate compound increases SMN protein levels relative to cells contacted with said agent but not contacted with the candidate compound, wherein an increase in SMN protein levels identifies the combination as a combination useful for the treatment of a neurodegenerative disease.

27. The method of claim 26, wherein said cells are mammalian cells.

28. The method of claim 27, wherein said cells are human cells.

29. The method of claim 28, wherein said cells are derived from a patient diagnosed as having SMA.

30. A composition comprising:
(a) a first agent selected from the agents of Table 1; and
(b) a second agent useful for treating a neurodegenerative disease, wherein said first agent and said second agent are present in amounts that, when administered to a patient in need thereof, are sufficient to increase SMN protein levels in a patient having SMA.

31. The composition of claim 30, further comprising one or more additional agents selected from Table 1.

32. The composition of claim 30, wherein said composition is formulated for oral administration.

33. The composition of claim 30, wherein said composition is formulated for systemic administration.

34. A method for increasing SMN protein levels in a patient having SMA, said method comprising administering to said patient one or more agents selected from the agents of Table 1 in an amount sufficient to increase SMN protein levels in said patient.

35. A method for increasing SMN protein levels in a patient having SMA, said method comprising administering to said patient at least two different agents selected from the agents Table 1, wherein the first and second agents are administered simultaneously or within 28 days of each other, in amounts that together are sufficient to increase SMN protein levels in said patient.

36. The method of claim 35, wherein said first and second agents are administered within 14 days of each other.

37. The method of claim 36, wherein said first and second agents are administered within 7 days of each other.

38. The method of claim 37, wherein said first and second agents are administered within 24 hours of each other.

39. The method of claim 35, further comprising administering to said patient an additional agent useful for treating a neurodegenerative disease, wherein the agent or agents from Table 1 and said additional therapeutic agents are present in amounts that, when administered to said patient, are sufficient to increase SMN protein levels.

40. The method of claim 39, wherein said agent or agents from Table 1 and said additional agent are administered within 14 days of each other.

41. The method of claim 39, wherein said agent or agents from Table 1 and/or said additional agent are administered orally or systemically.

42. A method of identifying a combination that increases SMN protein levels, said method comprising the steps of:
(a) contacting cells with a combination comprising an agent selected from the agents of Table 1 and a candidate compound; and
(b) determining whether the combination of said agent and said candidate compound increases SMN protein levels relative to cells contacted with said agent but not contacted with the candidate compound, wherein an increase in SMN protein levels identifies the combination as a combination that increases SMN protein levels.

43. The method of claim 42, wherein said cells are mammalian cells.

44. The method of claim 43, wherein said cells are human cells.

45. The method of claim 44, wherein said cells are derived from a patient diagnosed as having SMA.
